(19) United States
(12) Patent Application Publication
(10) Pub. No.: US 2010/0004235 A1
(43) Pub. Date: Jan. 7, 2010

(54) Heterocyclic Substituted, Anellated Pyrazole Derivative and its uses

(75) Inventors: Hartmut Schirok, Wuppertal (DE); Nils Griebenow, Dormagen (DE); Chantal Fürstner, Mulheim (DE); Joachim Mittendorf, Wuppertal (DE); Johannes-Peter Stasch, Solingen (DE); Frank Wunder, Wuppertal (DE); Karl-Heinz Schlemmer, Wuppertal (DE); Stefan Heitmeier, Wulfrath (DE); Friederike Stoll, Dusseldorf (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER HEALTHCARE AG, GERMANY (DE)

(21) Appl. No.: 12/298,532

(22) PCT Filed: Apr. 17, 2007
(86) PCT No.: PCT/EP07/03342

§ 371 Date: Jun. 4, 2009

(30) Foreign Application Priority Data
Apr. 27, 2006 (DE) 102006020327.5

(51) Int. Cl. A61K 31/5377; C07D 471/02; A61K 31/437; C07D 413/14; A61P 9/00

(52) U.S. Cl. 514/234.2; 546/119; 514/303; 544/127

(57) ABSTRACT

The present application relates to novel heterocyclyl-substituted fused pyrazole derivatives, to processes for their preparation, to their use, alone or in combination, for the treatment and/or prevention of diseases and to their use for preparing medicaments for the treatment and/or prevention of diseases, in particular for the treatment and/or prevention of cardiovascular disorders.

The present application relates to novel heterocyclyl-substituted fused pyrazole derivatives, to processes for their preparation, to their use, alone or in combination, for the treatment and/or prevention of diseases and to their use for preparing medicaments for the treatment and/or prevention of diseases, in particular for the treatment and/or prevention of cardiovascular disorders.

One of the most important cellular transmission systems in mammalian cells is cyclic guanosine monophosphate (cGMP). Together with nitric oxide (NO), which is released from the endothelium and transmits hormonal and mechanical signals, it forms the NO/cGMP system. Guanylate cyclases catalyze the biosynthesis of cGMP from guanosine triphosphate (GTP). The representatives of this family disclosed to date can be divided both according to structural features and according to the type of ligands into two groups: the particulate guanylate cyclases which can be stimulated by natriuretic peptides, and the soluble guanylate cyclases which can be stimulated by NO. The soluble guanylate cyclases consist of two subunits and very probably contain one heme per heterodimer, which is part of the regulatory site. The latter is of central importance for the mechanism of activation. NO is able to bind to the iron atom of heme and thus markedly increase the activity of the enzyme. Heme-free preparations cannot, by contrast, be stimulated by NO. Carbon monoxide (CO) is also able to attach to the central iron atom of heme, but the stimulation by CO is distinctly less than that by NO.

Through the production of cGMP and the regulation, resulting therefrom, of phosphodiesterases, ion channels and protein kinases, guanylate cyclase plays a crucial part in various physiological processes, in particular in the relaxation and proliferation of smooth muscle cells, in platelet aggregation and adhesion and in neuronal signal transmission, and in disorders caused by an impairment of the aforementioned processes. Under pathophysiological conditions, the NO/cGMP system may be suppressed, which may lead for example to high blood pressure, platelet activation, increased cellular proliferation, endothelial dysfunction, atherosclerosis, angina pectoris, heart failure, myocardial infarction, thromboses, stroke and sexual dysfunction.

A possible way of treating such disorders which is independent of NO and aims at influencing the cGMP signaling pathway in organisms is a promising approach because of the high efficiency and few side effects which are to be expected.

Compounds, such as organic nitrates, whose effect is based on NO have to date been exclusively used for the therapeutic stimulation of soluble guanylate cyclase. NO is produced by bioconversion and activates soluble guanylate cyclase by attaching to the central iron atom of heme. Besides the side effects, the development of tolerance is one of the crucial disadvantages of this mode of treatment.

Some substances which directly stimulate soluble guanylate cyclase, i.e. without previous release of NO, have been described in recent years, such as, for example, 3-(5′-hydroxymethyl-2′-furyl)-1-benzylindazole [YC-1, Wu et al., Blood 84 (1994), 4226; Mülsch et al., Brit. J. Pharmacol. 120 (1997), 681], fatty acids [Goldberg et al., J. Biol. Chem. 252 (1977), 1279], diphenyliodonium hexafluorophosphate [Pettibone et al., Eur. J. Pharmacol. 116 (1985), 307], isoliquiritigenin [Yu et al., Brit. J. Pharmacol. 114 (1995), 1587] and various substituted pyrazole derivatives (WO 98/16223).

Further heterocyclically substituted fused pyrazole derivatives are described inter alia in WO 98/16507, WO 98/23619 and WO 00/06569 as stimulators of soluble guanylate cyclase. However, it has been found that these compounds have disadvantages with respect to their in vivo properties, such as, for example, their behavior in the liver, their pharmacokinetic behavior, their dose-activity relationship and/or their metabolic path.

Certain heterocyclically substituted indazoles and their use for blocking voltage-gated sodium channels in glaucoma and multiple sclerosis are claimed in WO 01/57024. Furthermore, WO 2005/030121 claims heterocyclically substituted fused pyrazole derivatives for treating tumor disorders.

A. Straub et al., Bioorg. Med. Chem. Lett. 11, 781-784 (2001), report a moderate vessel-relaxing effect of the compound 1-(2-fluorobenzyl)-3-(1H-tetrazol-5-yl)-1H-pyrazolo[3,4-b]pyridine. Various 1-benzyl-3-(1H-tetrazol-5-yl)-1H-indazole derivatives are known from G. Corsi et al., J. Med. Chem. 19 (6), 778-783 (1976).

It was an object of the present invention to provide novel substances which act as stimulators of soluble guanylate cyclase and, compared to the compounds known from the prior art, have an improved therapeutic profile.

This object is achieved by the compounds described in the present invention. These compounds are distinguished by a fused pyrazole core structure which, in the 3-position, is linked to certain NH-acidic heterocycles.

Specifically, the present invention relates to compounds of the general formula (I)

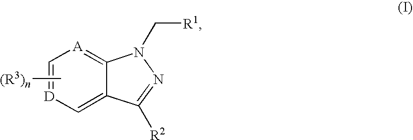

in which
A represents CH, CR³ or N,
D represents CH, CR³ or N if A is N and represents CH or CR³ if A is CH or CR³,
R¹ represents phenyl, pyridyl, furyl, thienyl, thiazolyl, oxazolyl, isothiazolyl or isoxazolyl, each of which may be substituted up to two times by identical or different substituents from the group consisting of halogen, cyano, (C₁-C₄)-alkyl, trifluoromethyl and (C₂-C₄)-alkynyl,
   or
   represents (C₅-C₇)-cycloalkyl which may be substituted up to two times by identical or different substituents from the group consisting of fluorine and (C₁-C₄)-alkyl,
R² represents a group of the formula

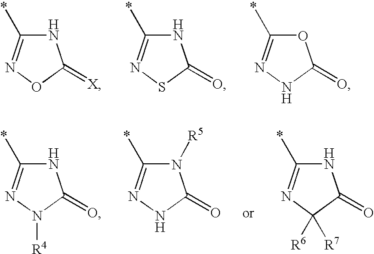

where
* represents the point of attachment to the pyrazole ring,
X represents O or S,
R⁴ represents hydrogen, (C₁-C₆)-alkyl or (C₃-C₇)-cycloalkyl,
   where (C₁-C₆)-alkyl may be substituted up to five times by fluorine and up to two times by identical or different substituents from the group consisting of (C₃-C₇)-cycloalkyl, hydroxyl, (C₁-C₄)-alkoxy, trifluoromethoxy, (C₁-C₄)-acyloxy, amino, mono-(C₁-C₄)-alkylamino, di-(C₁-C₄)-alkylamino, (C₁-C₄)-acylamino, hydroxycarbonyl, (C₁-C₄)-alkoxycarbonyl, a 5- or 6-membered heterocycle and a group of the formula —C(═O)—NR⁸R⁹, where
   R⁸ and R⁹ independently of one another represent hydrogen or (C₁-C₄)-alkyl,
R⁵ represents (C₁-C₄)-alkyl which may be substituted by hydroxyl, (C₁-C₄)-alkoxy, amino, mono-(C₁-C₄)-alkylamino, di-(C₁-C₄)-alkylamino or up to three times by fluorine,
R⁶ has the meaning of R⁴ given above,
R⁷ represents hydrogen or (C₁-C₄)-alkyl
or
R⁶ and R⁷ together with the carbon atom to which they are attached form a spiral-linked 3- to 6-membered cycloalkyl ring,
R³ represents a substituent selected from the group consisting of halogen, cyano, (C₁-C₄)-alkyl, trifluoromethyl, amino, (C₁-C₄)-alkoxy and trifluoromethoxy
and
n represents the number 0, 1 or 2,
   where, if the substituent R³ is present more than once, its meanings may be identical or different,
      and their salts, solvates and solvates of the salts.

Compounds according to the invention are the compounds of the formula (I) and the salts, solvates and solvates of the salts thereof, the compounds which are encompassed by formula (I) and are of the formulae mentioned hereinafter, and the salts, solvates and solvates of the salts thereof, and the compounds which are encompassed by formula (I) and are mentioned hereinafter as exemplary embodiments, and the salts, solvates and solvates of the salts thereof, insofar as the compounds encompassed by formula (I) and mentioned hereinafter are not already salts, solvates and solvates of the salts.

The compounds according to the invention may, depending on their structure, exist in stereoisomeric forms (enantiomers, diastereomers). The invention therefore relates to the enantiomers or diastereomers and respective mixtures thereof. The stereoisomerically pure constituents can be isolated in a known manner from such mixtures of enantiomers and/or diastereomers.

Where the compounds according to the invention can occur in tautomeric forms, the present invention encompasses all tautomeric forms.

Salts preferred for the purposes of the present invention are physiologically acceptable salts of the compounds according to the invention. However, salts which are themselves unsuitable for pharmaceutical applications but can be used for example for isolating or purifying the compounds according to the invention are also encompassed.

Physiologically acceptable salts of the compounds according to the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases such as, for example and preferably, alkali metal salts (e.g. sodium and potassium salts), alkaline earth metal salts (e.g. calcium and magnesium salts) and ammonium salts derived from ammonia or organic amines having 1 to 16 C atoms, such as, for example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methyl-morpholine, arginine, lysine, ethylenediamine and N-methylpiperidine.

Solvates refer for the purposes of the invention to those forms of the compounds according to the invention which form a complex in the solid or liquid state through coordination with solvent molecules. Hydrates are a specific form of solvates in which the coordination takes place with water. Solvates preferred in the context of the present invention are hydrates.

The present invention also encompasses prodrugs of the compounds according to the invention. The term “prodrugs” encompasses compounds which themselves may be biologically active or inactive but are converted during their residence time in the body into compounds according to the invention (for example by metabolism or hydrolysis).

In the context of the present invention, the substituents have the following meaning unless otherwise specified:

(C₁-C₈)-Alkyl, (C₁-C₆)-alkyl and (C₁-C₄)-alkyl are in the context of the invention a straight-chain or branched alkyl radical having 1 to 8, 1 to 6 and 1 to 4 carbon atoms, respectively. Preference is given to a straight-chain or branched alkyl radical having 1 to 4 carbon atoms. The following radicals may be mentioned by way of example and by way of preference: methyl, ethyl, n-propyl, isopropyl, n-butyl, isobutyl, sec-butyl, tert-butyl, 1-ethylpropyl, n-pentyl and n-hexyl.

(C₂-C₄)-Alkynyl is in the context of the invention a straight-chain or branched alkynyl radical having 2 to 4 carbon atoms and a triple bond. Preference is given to a straight-chain alkynyl radical having 2 to 4 carbon atoms. The following radicals may be mentioned by way of example and by way of preference: ethynyl, n-prop-1-yn-1-yl, n-prop-2-yn-1-yl, n-but-1-yn-1-yl, n-but-2-yn-1-yl and n-but-3-yn-1-yl.

(C₁-C₄)-Alkoxy is in the context of the invention a straight-chain or branched alkoxy radical having 1 to 4 carbon atoms. The following radicals may be mentioned by way of example and by way of preference: methoxy, ethoxy, n-propoxy, isopropoxy, n-butoxy and tert-butoxy.

(C₁-C₄)-Alkoxycarbonyl is in the context of the invention a straight-chain or branched alkoxy radical having 1 to 4 carbon atoms which is attached via a carbonyl group. The following radicals may be mentioned by way of example and by way of preference: methoxycarbonyl, ethoxycarbonyl, n-propoxycarbonyl, isopropoxycarbonyl and tert-butoxycarbonyl.

Mono-(C₁-C₄)-alkylamino and mono-(C₁-C₃)-alkylamino are in the context of the invention an amino group having a straight-chain or branched alkyl substituent having 1 to 4 and 1 to 3 carbon atoms, respectively. The following radicals may be mentioned by way of example and by way of preference: methylamino, ethylamino, n-propylamino, isopropylamino, n-butylamino and tert-butylamino.

Di-(C₁-C₄)-alkylamino and di-(C₁-C₃)-alkylamino are in the context of the invention an amino group having two identical or different straight-chain or branched alkyl substituents having in each case 1 to 4 and 1 to 3 carbon atoms, respectively. The following radicals may be mentioned by way of example and by way of preference: N,N-dimethylamino, N,N-diethylamino, N-ethyl-N-methylamino, N-methyl-N-n-propylamino, N-isopropyl-N-n-propylamino, N,N-diisopropylamino, N-n-butyl-N-methylamino and N-tert-butyl-N-methylamino.

(C₁-C₄)-Acyl[(C₁-C₄)-alkanoyl] is in the context of the invention a straight-chain or branched alkyl radical having 1 to 4 carbon atoms which carries a doubly attached oxygen atom in the 1-position and is attached via the 1-position. The following radicals may be mentioned by way of example and by way of preference: formyl, acetyl, propionyl, n-butyryl and isobutyryl.

(C₁-C₄)-Acylamino is in the context of the invention an amino group having a straight-chain or branched acyl substituent which has 1 to 4 carbon atoms and is attached via the carbonyl group to the nitrogen atom. The following radicals may be mentioned by way of example and by way of preference: formamido, acetamido, propionamido, n-butyramido and isobutyramido.

(C₁-C₄)-Acyloxy is in the context of the invention a straight-chain or branched alkyl radical having 1 to 4 carbon atoms which carries a doubly attached oxygen atom in the 1-position and is attached in the 1-position via a further oxygen atom. The following radicals may be mentioned by way of example and by way of preference: acetoxy, propionoxy, n-butyroxy and isobutyroxy.

(C₃-C₇)-Cycloalkyl, (C₃-C₆)-cycloalkyl and (C₅-C₇)-cycloalkyl are in the context of the invention a monocyclic saturated cycloalkyl group having 3 to 7, 3 to 6 and 5 to 7 ring carbon atoms, respectively. The following radicals may be mentioned by way of example and by way of preference: cyclopropyl, cyclobutyl, cyclopentyl, cyclohexyl and cycloheptyl.

A 5- or 6-membered heterocycle is in the context of the invention a saturated heterocycle having a total of 5 or 6 ring atoms which contains one or two ring heteroatoms from the group consisting of N, O and S and which is attached via a ring carbon atom or, if appropriate, a ring nitrogen atom. Preference is given to a 5- or 6-membered heterocycle having one or two ring heteroatoms from the group consisting of N and O. Examples which may be mentioned are: pyrrolidinyl, pyrazolidinyl, tetrahydrofuranyl, piperidinyl, piperazinyl, tetrahydropyranyl, morpholinyl and thiomorpholinyl. Preference is given to pyrrolidinyl, tetrahydrofuranyl, piperidinyl, piperazinyl and morpholinyl.

Halogen includes in the context of the invention fluorine, chlorine, bromine and iodine. Preference is given to chlorine or fluorine.

If radicals in the compounds according to the invention are substituted, the radicals may, unless otherwise specified, be substituted one or more times. In the context of the present invention, all radicals which occur more than once have a mutually independent meaning. Substitution by one, two or three identical or different substituents is preferred. Substitution by one substituent is very particularly preferred.

Preference is given in the context of the present invention to compounds of the formula (I) in which
A represents N.

Preference is given in the context of the present invention also to compounds of the formula (I) in which
D represents CH.

Preference is given in the context of the present invention likewise to compounds of the formula (I) in which
R¹ represents phenyl or thienyl, each of which may be substituted up to two times by identical or different substituents from the group consisting of fluorine, chlorine, cyano, methyl and trifluoromethyl,
   or
   represents cyclohexyl or cycloheptyl, each of which may be substituted up to two times by identical or different substituents from the group consisting of fluorine and methyl.

Particular preference is given in the context of the present invention to compounds of the formula (I), in which
A represents N,
D represents CH,
R¹ represents phenyl or thienyl, each of which is mono- or disubstituted by identical or different substituents from the group consisting of fluorine, chlorine, cyano, methyl and trifluoromethyl, or represents cycloheptyl,
R² represents a group of the formula

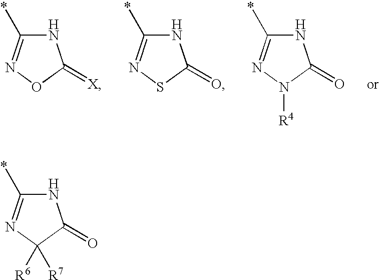

where
* represents the point of attachment to the pyrazole ring,
X represents O or S,
R⁴ represents hydrogen or (C₁-C₄)-alkyl,
   where (C₁-C₄)-alkyl may be substituted up to five times by fluorine and up to two times by identical or different substituents from the group consisting of (C₃-C₆)-cycloalkyl, hydroxyl, methoxy, ethoxy, trifluoromethoxy, acetoxy, amino, mono-(C₁-C₃)-alkylamino, di-(C₁-C₃)-alkylamino, acetylamino, (C₁-C₄)-alkoxycarbonyl, hydroxycarbonyl, morpholino, piperidino, pyrrolidino and a group of the formula —C(═O)—NR⁸R⁹, where
      R⁸ and R⁹ independently of one another represents hydrogen or (C₁-C₄)-alkyl,
R⁶ has the meaning of R⁴ given above
and
R⁷ represents hydrogen,
R³ represents a substituent selected from the group consisting of fluorine, chlorine, methyl, trifluoromethyl, amino, methoxy and trifluoromethoxy
   and
n represents the number 0 or 1,
   and their salts, solvates and solvates of the salts.

Very particular preference is given in the context of the present invention to compounds of the formula (I), in which
A represents N,
D represents CH,
R¹ represents phenyl which is mono- or disubstituted by fluorine,
R² represents a group of the formula

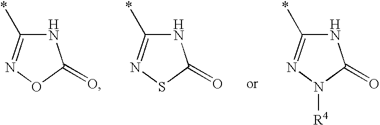

where
* represents the point of attachment to the pyrazole ring
and
R⁴ represents hydrogen or (C₁-C₄)-alkyl which may be substituted up to three times by fluorine,
   and
n represents the number 0,
   and their salts, solvates and solvates of the salts.

The definitions of radicals indicated specifically in the respective combinations or preferred combinations of radicals are replaced as desired irrespective of the particular combinations indicated for the radicals also by definitions of radicals of other combinations.

Combinations of two or more of the abovementioned preferred ranges are very particularly preferred.

The invention furthermore provides a process for preparing the compounds of the formula (I) according to the invention, characterized in that either

[A] a compound of the formula (II)

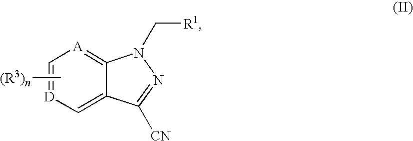

in which A, D, R¹, R³ and n each have the meanings given above,
is initially converted with hydroxylamine into an N′-hydroxyamidine of the formula (III)

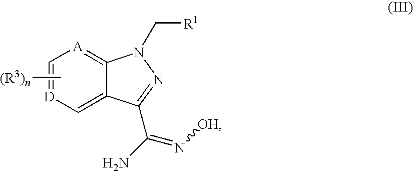

in which A, D, R¹, R³ and n each have the meanings given above,
and this is then converted
[A-1] in the presence of a base with phosgene, a phosgene derivative, such as diphosgene or triphosgene, N,N-carbonyldiimidazole or a chloroformate of the formula (IV)

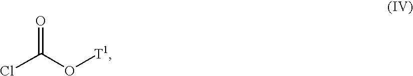

in which
T¹ represents (C₁-C₈)-alkyl,
into a compound of the formula (I-A)

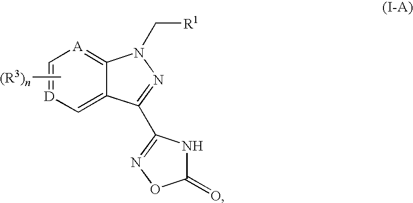

in which A, D, R¹, R³ and n each have the meanings given above,
or
[A-2] in the presence of a base with thiophosgene or N,N′-thiocarbonyldiimidazole into a compound of the formula (I-B)

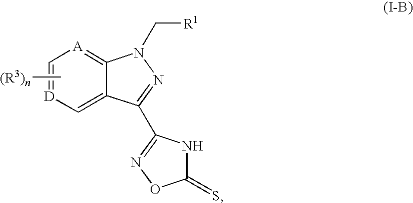

in which A, D, R¹, R³ and n each have the meanings given above,
or
[A-3] initially with N,N′-thiocarbonyldiimidazole and then with boron trifluoride into a compound of the formula (I-C)

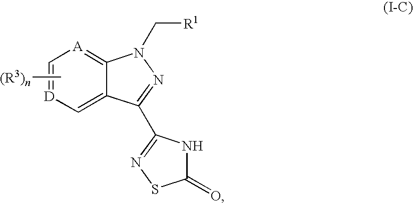

in which A, D, R¹, R³ and n each have the meanings given above,
   or
   [B] a compound of the formula (V)

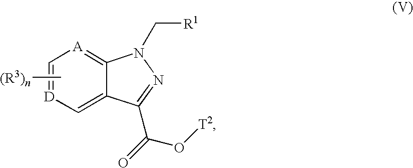

in which A, D, R¹, R³ and n each have the meanings given above
and
T² represents methyl or ethyl,
is initially converted with hydrazine into a compound of the formula (VI)

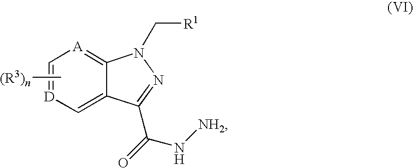

in which A, D, R¹, R³ and n each have the meanings given above,
and this is then converted
[B-1] with phosgene, a phosgene derivative, such as diphosgene or triphosgene, or N,N′-carbonyldiimidazole into a compound of the formula (I-D)

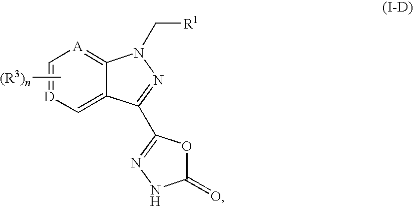

in which A, D, R¹, R³ and n each have the meanings given above,
or
[B-2] with an isocyanate of the formula (VII)

R^{5A}—N═C═O (VII),
in which
R^{5A} has the meaning of R⁵ given above or represents 2,4-dimethoxybenzyl, into a compound of the formula (VIII)

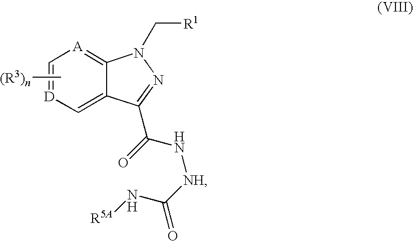

in which A, D, R¹, R³, R^{5A} and n each have the meanings given above,
and then cyclized with the aid of a base to a compound of the formula (I-E)

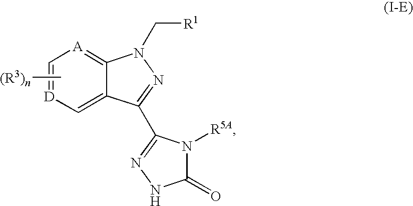

in which A, D, R¹, R³, R^{5A} and n each have the meanings given above,
and in the variant where R^{5A} represents 2,4-dimethoxybenzyl, the corresponding compound of the formula (I-E) is then converted in the presence of a base with a compound of the formula (IX)

R^{4A}—X¹ (IX),
in which
R^{4A} has the meaning of R⁴ given in any of claims 1 to 3, but does not represent hydrogen,
and
X¹ represents a leaving group, such as halogen, mesylate, tosylate or triflate, to a compound of the formula (X)

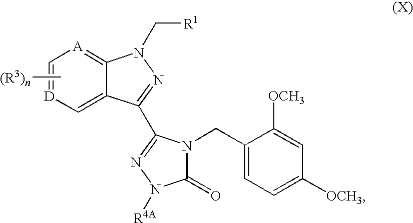

in which A, D, R¹, R³, R^{4A} and n each have the meanings given above,
and the 2,4-dimethoxybenzyl group is then removed with the aid of an acid, giving a compound of the formula (I-F)

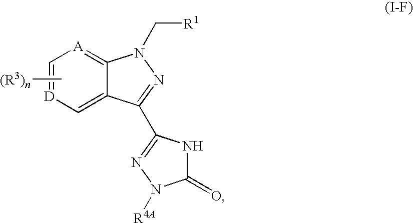

in which A, D, R¹, R³, R^{4A} and n each have the meanings given above,
   or
   [C] a compound of the formula (XI)

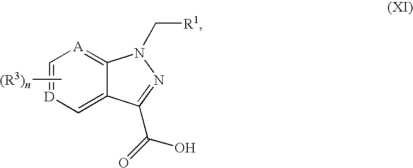

in which A, D, R¹, R³ and n each have the meanings given above,
is initially coupled with a compound of the formula (XII)

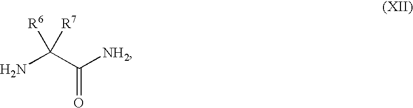

in which R⁶ and R⁷ have the meanings given above,
to give a compound of the formula (XIII)

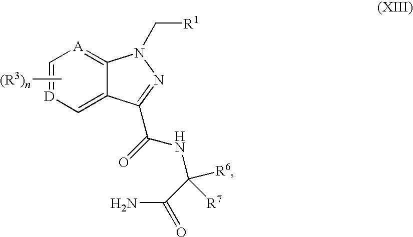

in which A, D, R¹, R³, R⁶, R⁷ and n each have the meanings given above,
and this is then cyclized with phosphoryl chloride to a compound of the formula (I-G)

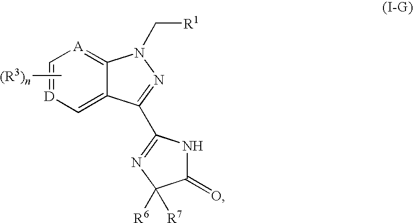

in which A, D, R¹, R³, R⁶, R⁷ and n each have the meanings given above,
   and the resulting compounds according to the invention are, if appropriate, converted with the appropriate (i) solvents and/or (ii) acids or bases into their solvates, salts and/or solvates of the salts.

Inert solvents for the process steps (II)→(III), (III)→(I-A), (III)→(I-B), (VI)→(I-D), (VI)+(VII)→(VIII) and (I-E)+(IX)→(X) are, for example, ethers, such as diethyl ether, methyl tert-butyl ether, dioxane, tetrahydrofuran, glycol dimethyl ether or diethylene glycol dimethyl ether, hydrocarbons, such as benzene, toluene, xylene, hexane, cyclohexane or mineral oil fractions, halogenated hydrocarbons, such as dichloromethane, trichloromethane, carbon tetrachloride, 1,2-dichloroethane, trichloroethane, tetrachloroethane, trichloroethylene, chlorobenzene or chlorotoluene, or other solvents, such as dimethyl sulfoxide (DMSO), dimethylformamide (DMF), N,N′-dimethylpropyleneurea (DMPU), N-methylpyrrolidone (NMP) or acetonitrile. It is also possible to use mixtures of the solvents mentioned. Preference is given to using dichloromethane, tetrahydrofuran, dimethylformamide, dimethyl sulfoxide, acetonitrile, toluene, xylene or mixtures of these solvents.

The process step (V)→(VI) is preferably carried out in an alcohol, such as methanol, ethanol, n-propanol, isopropanol, n-butanol or tert-butanol, in an ether, such as diethyl ether, methyl tert-butyl ether, dioxane, tetrahydrofuran, glycol dimethyl ether or diethylene glycol dimethyl ether, or in mixtures of these as solvent. Preference is given to using a mixture of methanol and tetrahydrofuran.

The process step (VIII)→(I-E) is preferably carried out in water or in a solvent such as dimethylformamide or dimethyl sulfoxide.

Suitable bases for the process steps (III)→(I-A), (III)→(I-B), (VIII)→(I-E) and (I-E)+(IX)→(X) are customary inorganic or organic bases. These preferably include alkali metal hydroxides, such as, for example, lithium hydroxide, sodium hydroxide or potassium hydroxide, alkali metal or alkaline earth metal carbonates, such as lithium carbonate, sodium carbonate, potassium carbonate, calcium carbonate or cesium carbonate, alkali metal hydrides, such as sodium hydride or potassium hydride, amides, such as lithium bis(trimethylsilyl)amide or potassium bis(trimethylsilyl)amide or lithium diisopropylamide, or organic amines, such as triethylamine, N-methylmorpholine, N-methylpiperidine, N,N-diisopropylethylamine, pyridine, 4-N,N-dimethylaminopyridine, 1,5-diazabicyclo[4.3.0]non-5-ene (DBN), 1,4-diazabicyclo[2.2.2]octane (DABCO®) or 1,8-diazabicyclo[5.4.0]undec-7-ene (DBU). Preference is given to using cesium carbonate, sodium hydride or pyridine.

The reactions mentioned are generally, depending on the reactivity of the reaction partners involved, carried out in a temperature range of from 0° C. to +140° C. The reactions can be carried out at atmospheric, elevated or reduced pressure (for example from 0.5 to 5 bar). In general, the reactions are carried out at atmospheric pressure.

The reaction sequence (III)→(I-C) is carried out analogously to a process described in the literature [see Y. Kohara et al., J. Heterocycl. Chem. 37, 1419 (2000)].

Suitable for removing the 2,4-dimethoxybenzyl protective group in process step (X)→(I-F) are in particular acids such as p-toluenesulfonic acid, trifluoroacetic acid or sulfuric acid. The reaction is preferably carried out in toluene or acetic acid in a temperature range of from +20° C. to +120° C.

Inert solvents for the process step (XI)+(XII)→(XIII) are, for example, ethers, such as diethyl ether, methyl tert-butyl ether, dioxane, tetrahydrofuran, glycol dimethyl ether or diethylene glycol dimethyl ether, hydrocarbons, such as benzene, toluene, xylene, hexane, cyclohexane or mineral oil fractions, halogenated hydrocarbons, such as dichloromethane, trichloromethane, carbon tetrachloride, 1,2-dichloroethane, trichloroethylene or chlorobenzene, or other solvents, such as ethyl acetate, acetone, dimethyl sulfoxide (DMSO), dimethylformamide (DMF), N,N′-dimethylpropyleneurea (DMPU), N-methylpyrrolidone (NMP), acetonitrile or pyridine. It is also possible to use mixtures of the solvents mentioned. Preference is given to dichloromethane, tetrahydrofuran, dimethylformamide, acetonitrile or mixtures of these solvents.

Suitable condensing agents for the amide formation in process step (XI)+(XII)→(XIII) are, for example, carbodiimides, such as N,N′-diethyl-, N,N′-dipropyl-, N,N′-diisopropyl-, N,N′-dicyclohexylcarbodiimide (DCC), N-(3-dimethylaminoisopropyl)-N′-ethylcarbodiimide hydrochloride (EDC), or phosgene derivatives, such as N,N′-carbonyldiimidazole, or 1,2-oxazolium compounds, such as 2-ethyl-5-phenyl-1,2-oxazolium 3-sulfate or 2-tert-butyl-5-methylisoxazolium perchlorate, or acylamino compounds, such as 2-ethoxy-1-ethoxycarbonyl-1,2-dihydroquinoline, or isobutyl chloroformate, propanephosphonic anhydride (PPA), diethyl cyanophosphonate, bis(2-oxo-3-oxazolidinyl)phosphoryl chloride, benzotriazol-1-yloxytris(dimethylamino)phosphonium hexafluorophosphate, benzotriazol-1-yloxytris(pyrrolidino)phosphonium hexafluorophosphate (Py-BOP), O-(benzotriazol-1-yl)-N,N,N′,N′-tetramethyluronium tetrafluoroborate (TBTU), O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), O-(7-azabenzotriazol-1-yl)-N,N,N′,N′-tetramethyluronium hexafluorophosphate (HATU) or O-(1H-6-chlorobenzotriazol-1-yl)-1,1,3,3-tetramethyluronium tetrafluoroborate (TCTU), if appropriate in combination with further auxiliaries, such as 1-hydroxylbenzotriazole (HOBt) or N-hydroxysuccinimide (HOSu), and also as bases alkali metal carbonates, for example sodium carbonate or potassium carbonate or sodium bicarbonate or potassium bicarbonate, or organic bases, such as trialkylamines, for example triethylamine, N-methylmorpholine, N-methylpiperidine or N,N-diisopropylethylamine. Preference is given to DCC or EDC, in each case in combination with HOBt and N,N-diisopropylethylamine.

The process step (XI)+(XII)→(XIII) is generally carried out in a temperature range of from −20° C. to +60° C., preferably at from 0° C. to +40° C. The reaction can be carried out at atmospheric, elevated or reduced pressure (for example from 0.5 to 5 bar). In general, the reaction is carried out at atmospheric pressure.

The cyclization in process step (XIII)→(I-G) can be carried out in excess phosphoryl chloride without further solvent or using a hydrocarbon, such as benzene, toluene, xylene, hexane or cyclohexane, or a halogenated hydrocarbon, such as dichloromethane, trichloromethane, carbon tetrachloride, 1,2-dichloroethane, trichloroethane, tetrachloroethane, trichloroethylene or chlorobenzene, as inert solvent.

The compounds of the formula (II) can be prepared by reacting a compound of the formula (XIV)

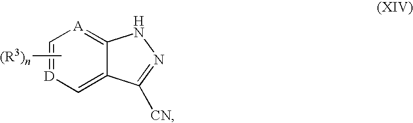

in which A, D, R³ and n each have the meanings given above,
in an inert solvent in the presence of a base with a compound of the formula (XV)

R¹—CH₂—X² (XV),

in which
R¹ has the meaning given above
and
X² represents a leaving group, such as halogen, mesylate, tosylate or triflate.

Inert solvents for the process step (XIV)+(XV)→(II) are, for example, ethers, such as diethyl ether, methyl tert-butyl ether, dioxane, tetrahydrofuran, glycol dimethyl ether or diethylene glycol dimethyl ether, hydrocarbons, such as benzene, toluene, xylene, hexane, cyclohexane or mineral oil fractions, halogenated hydrocarbons, such as dichloromethane, trichloromethane, carbon tetrachloride, 1,2-dichloroethane, trichloroethane, tetrachloroethane, trichloroethylene, chlorobenzene or chlorotoluene, or other solvents, such as dimethylformamide (DMF), dimethyl sulfoxide (DMSO), N,N′-dimethylpropyleneurea (DMPU), N-methylpyrrolidone (NMP), acetone, acetonitrile or pyridine. It is also possible to use mixtures of the solvents mentioned. Preference is given to using dimethylformamide.

Suitable bases for the process step (XIV)+(XV)→(II) are customary inorganic or organic bases. These preferably include alkali metal hydroxides, such as, for example, lithium hydroxide, sodium hydroxide or potassium hydroxide, alkali metal carbonates or alkaline earth metal carbonates, such as lithium carbonate, sodium carbonate, potassium carbonate, calcium carbonate or cesium carbonate, alkali metal alkoxides, such as sodium tert-butoxide or potassium tert-butoxide, alkali metal hydrides, such as sodium hydride or potassium hydride, amides, such as lithium bis(trimethylsilyl)amide or potassium bis(trimethylsilyl)amide or lithium diisopropylamide, organometallic compounds, such as butyllithium or phenyllithium, or organic amines, such as triethylamine, N-methylmolpholine, N-methylpiperidine, N,N-diisopropylethylamine or pyridine. Preference is given to using cesium carbonate.

The process step (XIV)+(XV)→(II) is generally carried out in a temperature range of from 0° C. to +100° C., preferably at from +20° C. to +50° C. The reaction can be carried out at atmospheric, elevated or reduced pressure (for example from 0.5 to 5 bar). In general, the reaction is carried out at atmospheric pressure.

The compounds of the formula (XIV) are known from the literature or can be prepared analogously to processes known from the literature [cf., for example, WO 00/06569; G. M. Shutske et al., J. Heterocycl. Chem. 34, 789 (1997); H. Salkowski, Chem. Ber. 17, 506 (1884), ibid., 22, 2139 (1889); M. M. Abdel-Khalik et al., Synthesis, 1166 (2000)]. The compound of the formula (XIV) in which A represents N, D represents CH and n represents 0 can also be obtained from 2-fluoropyridine (XVI)

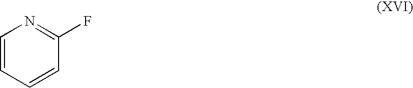

by acylation with trifluoroacetic ester and subsequent condensation with hydrazine to give 3-(trifluoromethyl)pyrazolopyridine of the formula (XVII)

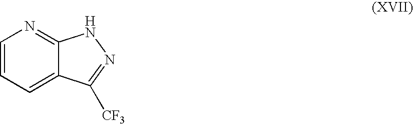

and subsequent reaction of (XVII) with ammonia (see reaction scheme 1).

The compounds of the formula (V) are known from the literature or can be prepared analogously to processes known from the literature [cf., for example, WO 00/06569; Corsi et al., J. Med. Chem. 19, 778, 781 (1976); H. Harada et al., Chem. Pharm. Bull. 43, 1912 (1995); K. Rehse et al., Arch. Pharm. 337, 311 (2004)]. For example, compounds of the formula (V) in which A represents N, D represents CH and n represents 0 can be obtained by reacting a hydrazine derivative of the formula (XVIII)

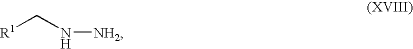

in which R¹ has the meaning given above,
with the sodium salt of a cyanopyruvic ester of the formula (XIX)

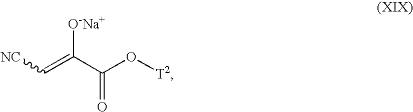

in which T² has the meaning given above,
to give the 5-aminopyrazole-3-carboxylic ester of the formula (XX)

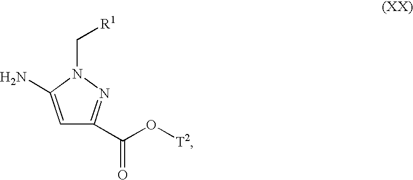

in which R¹ and T² have the meanings given above,
and subsequent condensation of (XX) with 3-dimethylaminoacrolein (see, for example, the preparation process described in WO 00/06569).

In a similar manner, it is possible to obtain, for example, compounds of the formula (II) in which A and D represent N, n represents 1 and R³ represents 4-amino by reacting the hydrazine derivative (XVIII) with tetracyanoethylene to give the 5-aminopyrazole-3,4-dicarbonitrile of the formula (XXI)

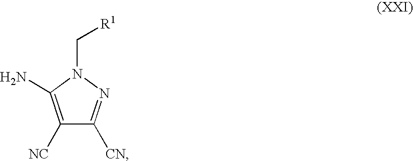

in which R¹ has the meaning given above,
and subsequent reaction of (XXI) with triethyl orthoformate and ammonia [see reaction scheme 4; cf., for example, F. Gatta et al., J. heterocycl. Chem. 26, 613 (1989)].

The compounds of the formula (XI) can be obtained by ester hydrolysis from the compounds of the formula (V).

The compounds of the formulae (IV), (VII), (IX), (XII), (XV), (XVI), (XVIII) and (XIX) are commercially available, known from the literature or can be prepared analogously to processes known from the literature.

The preparation of the compounds according to the invention can be illustrated by the synthesis schemes below:

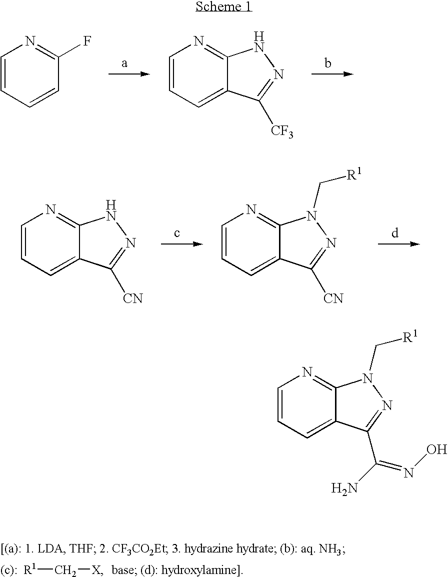

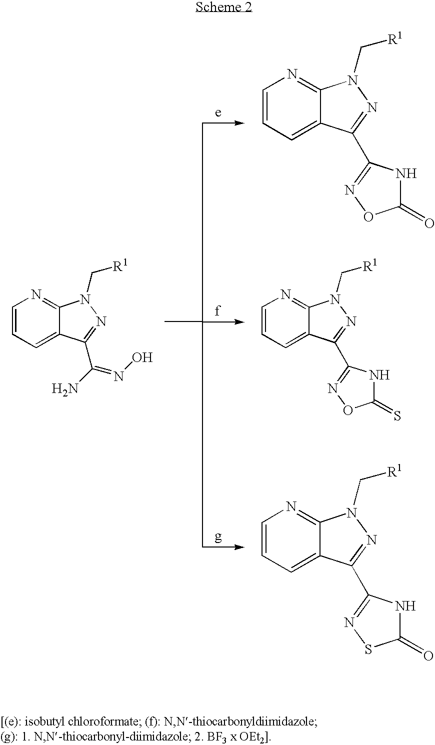

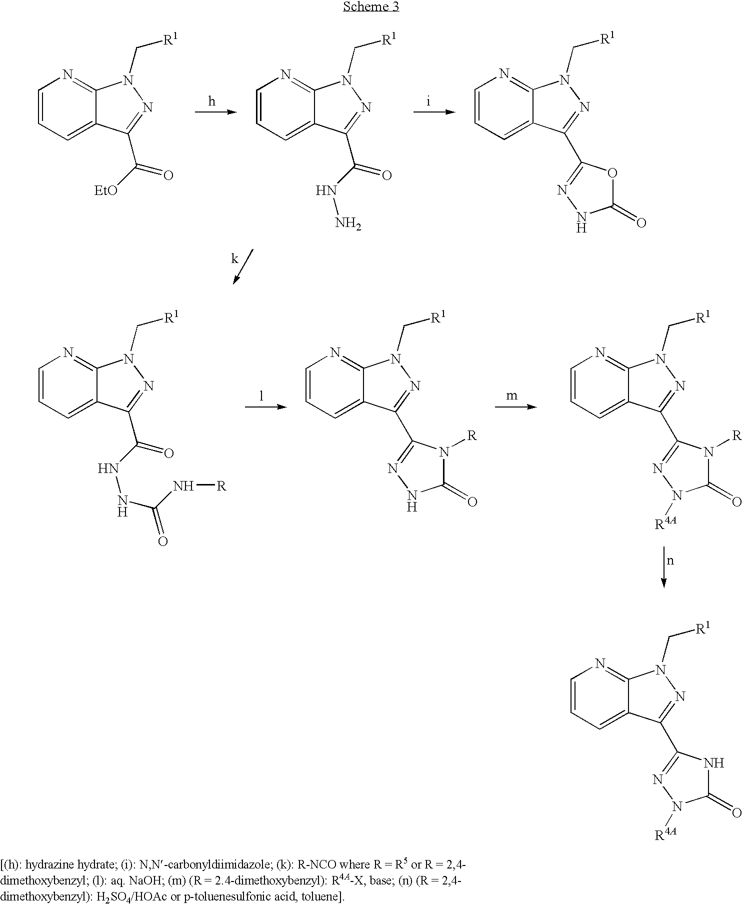

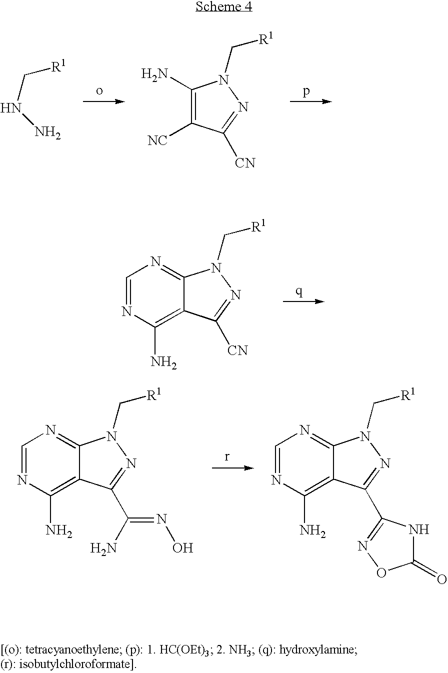

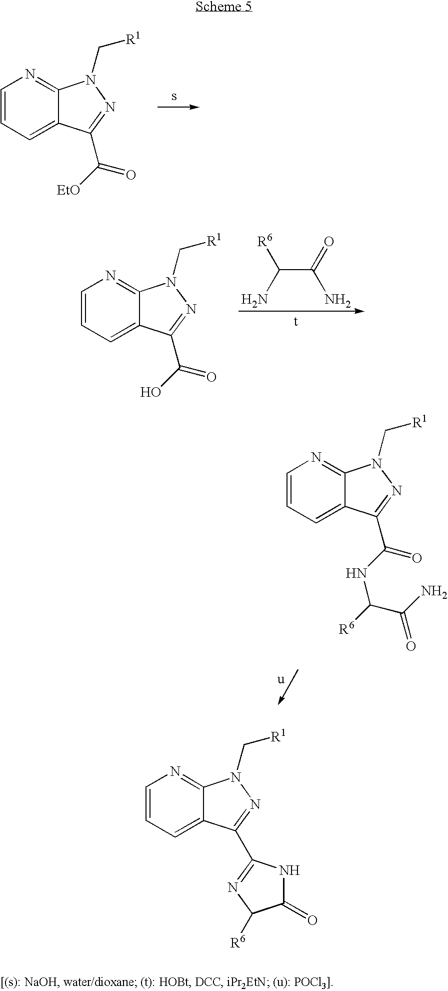

The compounds according to the invention have valuable pharmacological properties and can be used for the prevention and treatment of disorders in humans and animals.

The compounds according to the invention offer a further treatment alternative and enlarge pharmacy. Compared to the substances known from the prior art, the compounds according to the invention surprisingly have an improved therapeutic profile. An advantage of the compounds according to the invention is in particular their increased plasma concentration after oral administration.

The compounds according to the invention lead to vasorelaxation, to an inhibition of platelet aggregation and to a reduction in blood pressure, and also to an increase in coronary blood flow. These effects are mediated by direct stimulation of soluble guanylate cyclase and an increase in intracellular cGMP. Moreover, the compounds according to the invention enhance the effect of substances increasing the cGMP concentration, such as, for example, EDRF (endothelium-derived relaxing factor), NO donors, protoporphyrin IX, arachidonic acid or phenylhydrazine derivatives.

The compounds according to the invention can therefore be employed in medicaments for the treatment of cardiovascular disorders such as, for example, for the treatment of high blood pressure and heart failure, stable and unstable angina pectoris, pulmonary hypertension, peripheral and cardiac vascular disorders, arrhythmias, for the treatment of thromboembolic disorders and ischemias such as myocardial infarction, stroke, transistoric and ischemic attacks, disturbances of peripheral blood flow, reperfusion damage, prevention of restenoses as after thrombolysis therapies, percutaneous transluminal angioplasties (PTAs), percutaneous transluminal coronary angioplasties (PTCAs), bypass and for the treatment of arteriosclerosis, asthmatic disorders and diseases of the urogenital system such as, for example, prostate hypertrophy, erectile dysfunction, female sexual dysfunction, and incontinence, osteoporosis, glaucoma, and gastroparesis.

The compounds according to the invention can additionally be used for the treatment of primary and secondary Raynaud's phenomenon, of microcirculation impairments, claudication, peripheral and autonomic neuropathies, diabetic microangiopathies, diabetic retinopathy, diabetic ulcers on the extremities, gangrenes, CREST syndrome, erythematosis, onychomycosis, rheumatic disorders and for promoting wound healing.

The compounds according to the invention are furthermore suitable for the treatment of acute and chronic lung diseases, such as respiratory distress syndromes (ALI, ARDS) and chronic obstructive airway disorders (COPD), and also for the treatment of acute and chronic renal failure.

The compounds described in the present invention also represent active ingredients for controlling central nervous system diseases characterized by disturbances of the NO/cGMP system. They are suitable in particular for improving perception, concentration, learning or memory after cognitive impairments like those occurring in particular in association with situations/diseases/syndromes such as mild cognitive impairment, age-associated learning and memory impairments, age-associated memory losses, vascular dementia, craniocerebral trauma, stroke, dementia occurring after strokes (post stroke dementia), post-traumatic craniocerebral trauma, general concentration impairments, concentration impairments in children with learning and memory problems, Alzheimer's disease, Lewy body dementia, dementia with degeneration of the frontal lobes including Pick's syndrome, Parkinson's disease, progressive nuclear palsy, dementia with corticobasal degeneration, amyolateral sclerosis (ALS), Huntington's disease, multiple sclerosis, thalamic degeneration, Creutzfeld-Jacob dementia, HIV dementia, schizophrenia with dementia or Korsakoff's psychosis. They are also suitable for the treatment of central nervous system disorders such as states of anxiety, tension and depression, CNS-related sexual dysfunctions and sleep disturbances, and for controlling pathological disturbances of the intake of food, stimulants and addictive substances.

The compounds according to the invention are furthermore also suitable for controlling cerebral blood flow and thus represent effective agents for controlling migraine. They are also suitable for the prophylaxis and control of the sequelae of cerebral infarctions (Apoplexia cerebri) such as stroke, cerebral ischemias and craniocerebral trauma. The compounds according to the invention can likewise be employed for controlling states of pain.

In addition, the compounds according to the invention have an anti-inflammatory effect and can therefore be employed as anti-inflammatory agents.

The present invention further relates to the use of the compounds according to the invention for the treatment and/or prevention of disorders, especially of the aforementioned disorders.

The present invention further relates to the use of the compounds according to the invention for producing a medicament for the treatment and/or prevention of disorders, especially of the aforementioned disorders.

The present invention further relates to a method for the treatment and/or prevention of disorders, especially of the aforementioned disorders, by using an effective amount of at least one of the compounds according to the invention.

The compounds according to the invention can be employed alone or, if required, in combination with other active ingredients. The present invention further relates to medicaments comprising at least one of the compounds according to the invention and one or more further active ingredients, in particular for the treatment and/or prevention of the aforementioned disorders. Examples of suitable combination active ingredients which may be preferably mentioned are:
organic nitrates and NO donors such as, for example, sodium nitroprusside, nitroglycerin, isosorbide mononitrate, isosorbide dinitrate, molsidomine or SIN-1, and inhaled NO;
compounds which inhibit the breakdown of cyclic guanosine monophosphate (cGMP), such as, for example, inhibitors of phosphodiesterases (PDE) 1, 2 and/or 5, in particular PDE 5 inhibitors such as sildenafil, vardenafil and tadalafil;
agents having antithrombotic activity, for example and preferably from the group of platelet aggregation inhibitors, of anticoagulants or of profibrinolytic substances;
active ingredients which lower blood pressure, for example and preferably from the group of calcium antagonists, angiotensin AII antagonists, ACE inhibitors, endothelin antagonists, renin inhibitors, alpha-receptor blockers, beta-receptor blockers, mineralocorticoid receptor antagonists, and of diuretics; and/or
active ingredients which modify lipid metabolism, for example and preferably from the group of thyroid receptor agonists, cholesterol synthesis inhibitors such as, for example and preferably, HMG-CoA reductase inhibitors or squalene synthesis inhibitors, of ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein (a) antagonists.

Agents having antithrombotic activity preferably mean compounds from the group of platelet aggregation inhibitors, of anticoagulants or of profibrinolytic substances.

In a preferred embodiment of the invention, the compounds according to the invention are administered in combination with a platelet aggregation inhibitor such as, for example and preferably, aspirin, clopidogrel, ticlopidin or dipyridamole.

In a preferred embodiment of the invention, the compounds according to the invention are administered in combination with a thrombin inhibitor such as, for example and preferably, ximelagatran, melagatran, bivalirudin or clexane.

In a preferred embodiment of the invention, the compounds according to the invention are administered in combination with a GPIIb/IIIa antagonist such as, for example and preferably, tirofiban or abciximab.

In a preferred embodiment of the invention, the compounds according to the invention are administered in combination with a factor Xa inhibitor such as, for example and preferably, rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds according to the invention are administered in combination with heparin or with a low molecular weight (LMW) heparin derivative.

In a preferred embodiment of the invention, the compounds according to the invention are administered in combination with a vitamin K antagonist such as, for example and preferably, coumarin.

Agents which lower blood pressure preferably mean compounds from the group of calcium antagonists, angiotensin AII antagonists, ACE inhibitors, endothelin antagonists, renin inhibitors, alpha-receptor blockers, beta-receptor blockers, mineralocorticoid receptor antagonists, and of diuretics.

In a preferred embodiment of the invention, the compounds according to the invention are administered in combination with a calcium antagonist such as, for example and preferably, nifedipine, amlodipine, verapamil or diltiazem.

In a preferred embodiment of the invention, the compounds according to the invention are administered in combination with an alpha-1-receptor blocker such as, for example and preferably, prazosin.

In a preferred embodiment of the invention, the compounds according to the invention are administered in combination with a beta-receptor blocker such as, for example and preferably, propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds according to the invention are administered in combination with an angiotensin AII antagonist such as, for example and preferably, losartan, candesartan, valsartan, telmisartan or embursatan.

In a preferred embodiment of the invention, the compounds according to the invention are administered in combination with an ACE inhibitor such as, for example and preferably, enalapril, captopril, lisinopril, ramipril, delapril, fosinopril, quinopril, perindopril or trandopril.

In a preferred embodiment of the invention, the compounds according to the invention are administered in combination with an endothelin antagonist such as, for example and preferably, bosentan, darusentan, ambrisentan or sitaxsentan.

In a preferred embodiment of the invention, the compounds according to the invention are administered in combination with a renin inhibitor such as, for example and preferably, aliskiren, SPP-600 or SPP-800.

In a preferred embodiment of the invention, the compounds according to the invention are administered in combination with a mineralocorticoid receptor antagonist such as, for example and preferably, spironolactone or eplerenone.

In a preferred embodiment of the invention, the compounds according to the invention are administered in combination with a diuretic such as, for example and preferably, furosemide.

Agents which modify lipid metabolism preferably mean compounds from the group of CETP inhibitors, thyroid receptor agonists, cholesterol synthesis inhibitors such as HMG-CoA reductase inhibitors or squalene synthesis inhibitors, of ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and of lipoprotein(a) antagonists.

In a preferred embodiment of the invention, the compounds according to the invention are administered in combination with a CETP inhibitor such as, for example and preferably, torcetrapib (CP-529 414), JJT-705 or CETP vaccine (Avant).

In a preferred embodiment of the invention, the compounds according to the invention are administered in combination with a thyroid receptor agonist such as, for example and preferably, D-thyroxine, 3,5,3′-triiodothyronine (T3), CGS 23425 or axitirome (CGS 26214).

In a preferred embodiment of the invention, the compounds according to the invention are administered in combination with an HMG-CoA reductase inhibitor from the class of statins such as, for example and preferably, lovastatin, simvastatin, pravastatin, fluvastatin, atorvastatin, rosuvastatin, cerivastatin or pitavastatin.

In a preferred embodiment of the invention, the compounds according to the invention are administered in combination with a squalene synthesis inhibitor such as, for example and preferably, BMS-188494 or TAK-475.

In a preferred embodiment of the invention, the compounds according to the invention are administered in combination with an ACAT inhibitor such as, for example and preferably, avasimibe, melinamide, pactimibe, eflucimibe or SMP-797.

In a preferred embodiment of the invention, the compounds according to the invention are administered in combination with an MTP inhibitor such as, for example and preferably, implitapide, BMS-201038, R-103757 or JTT-130.

In a preferred embodiment of the invention, the compounds according to the invention are administered in combination with a PPAR-gamma agonist such as, for example and preferably, pioglitazone or rosiglitazone.

In a preferred embodiment of the invention, the compounds according to the invention are administered in combination with a PPAR-delta agonist such as, for example and preferably, GW 501516 or BAY 68-5042.

In a preferred embodiment of the invention, the compounds according to the invention are administered in combination with a cholesterol absorption inhibitor such as, for example and preferably, ezetimibe, tiqueside or pamaqueside.

In a preferred embodiment of the invention, the compounds according to the invention are administered in combination with a lipase inhibitor such as, for example and preferably, orlistat.

In a preferred embodiment of the invention, the compounds according to the invention are administered in combination with a polymeric bile acid adsorbent such as, for example and preferably, cholestyramine, colestipol, colesolvam, CholestaGel or colestimide.

In a preferred embodiment of the invention, the compounds according to the invention are administered in combination with a bile acid reabsorption inhibitor such as, for example and preferably, ASBT (=IBAT) inhibitors such as, for example, AZD-7806, S-8921, AK-105, BARI-1741, SC-435 or SC-635.

In a preferred embodiment of the invention, the compounds according to the invention are administered in combination with a lipoprotein (a) antagonist such as, for example and preferably, gemcabene calcium (CI-1027) or nicotinic acid.

The present invention further relates to medicaments which comprise at least one compound according to the invention, normally together with one or more inert, non-toxic, pharmaceutically suitable excipients, and to the use thereof for the aforementioned purposes.

The compounds according to the invention can act systemically and/or locally. For this purpose, they can be administered in a suitable way such as, for example, by the oral, parenteral, pulmonal, nasal, sublingual, lingual, buccal, rectal, dermal, transdermal, conjunctival, otic route or as implant stent.

The compounds according to the invention can be administered in administration forms suitable for these administration routes.

Suitable for oral administration are administration forms which function according to the prior art and deliver the compounds according to the invention rapidly and/or in modified fashion, and which contain the compounds according to the invention in crystalline and/or amorphized and/or dissolved form, such as, for example, tablets (uncoated or coated tablets, for example having enteric coatings or coatings which are insoluble or dissolve with a delay and control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, preparations for injection and infusion in the form of solutions, suspensions, emulsions, lyophilizates or sterile powders.

Suitable for the other administration routes are, for example, pharmaceutical forms for inhalation (inter alia powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (e.g. patches), milk, pastes, foams, dusting powders, implants or stents.

Oral or parenteral administration is preferred, especially oral administration.

The compounds according to the invention can be converted into the stated administration forms. This can take place in a manner known per se by mixing with inert, non-toxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colors (e.g. inorganic pigments such as, for example, iron oxides) and masking flavors and/or odors.

It has generally proved advantageous to administer on parenteral administration amounts of about 0.001 to 1 mg/kg, preferably about 0.01 to 0.5 mg/kg, of body weight to achieve effective results, and on oral administration the dosage is about 0.01 to 100 mg/kg, preferably about 0.01 to 20 mg/kg, and very particularly preferably 0.1 to 10 mg/kg, of body weight.

It may nevertheless be necessary where appropriate to deviate from the stated amounts, in particular as a function of the body weight, route of administration, individual response to the active ingredient, nature of the preparation and time or interval over which administration takes place. Thus, it may be sufficient in some cases to make do with less than the aforementioned minimum amount, whereas in other cases the stated upper limit must be exceeded. It may in the event of administration of larger amounts be advisable to divide these into a plurality of individual doses over the day.

The following exemplary embodiments illustrate the invention. The invention is not restricted to the examples.

The percentage data in the following tests and examples are, unless indicated otherwise, percentages by weight; parts are parts by weight. Solvent ratios, dilution ratios and concentration data for the liquid/liquid solutions are in each case based on volume.

### A. EXAMPLES

### Abbreviations

aq. Aqueous solution

### DCC N,N′-Dicyclohexylcarbodiimide

### DMF Dimethylformamide

DMSO Dimethyl sulfoxide
eq. Equivalent(s)
ESI Electrospray ionization (in MS)

### Et Ethyl

### h Hour(s)

HOAc Acetic acid

### HOBt 1-Hydroxy-1H-benzotriazole×H₂O

HPLC High-pressure, high-performance liquid chromatography

### iPr Isopropyl

LC/MS Liquid chromatography-coupled mass spectrometry
LDA Lithium diisopropylamide

### min Minute(s)

MS Mass spectrometry
NMR Nuclear magnetic spectrometry
RT Room temperature
Rₜ Retention time (in HPLC)

### THF Tetrahydrofuran

UV Ultraviolet spectrometry
v/v Volume to volume ratio (of a solution)

### LC/MS and HPLC Methods:

### Method 1 (LC/MS):

Instrument: Micromass Quattro LCZ with HPLC Agilent series 1100; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 208-400 nm.

### Method 2 (LC/MS):

MS instrument type: Micromass ZQ; HPLC instrument type: Waters Alliance 2795; column: Phenomenex Synergi 2μ Hydro-RP Mercury 20 mm×4 mm; mobile phase A: 111 of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 3 (LC/MS):

MS instrument type: Micromass ZQ; HPLC instrument type: HP 1100 series; UV DAD; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 4 (LC/MS):

MS instrument type: Micromass ZQ; HPLC instrument type: HP 1100 series; UV DAD; column: Phenomenex Gemini 3μ 30 mm×3.00 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 5 (Preparative HPLC):

Column: Grom-Sil 120 ODS-4HE, 10 μm, 250 mm×30 mm; mobile phase A: 0.1% formic acid in water, mobile phase B: acetonitrile; flow rate: 50 ml/min; gradient: 0-3 min 10% B, 3-27 min 10%→95% B, 27-34 min 95% B, 34-38 min 10% B.

### Method 6 (LC/MS):

Instrument: Micromass Platform LCZ with HPLC Agilent series 1100; column: Thermo Hypersil GOLD 3μ 20 mm×4 mm; mobile phase A: 1 l of water+0.5 ml of 50% strength formic acid, mobile phase B: 1 l of acetonitrile+0.5 ml of 50% strength formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

### Method 7 (Analytical HPLC):

Instrument: HP 1100 with DAD detection; column: Kromasil 100 RP-18, 60 mm×2.1 mm, 3.5 μm; mobile phase A: 5 ml of HClO₄ (70% strength)/liter of water, mobile phase B: acetonitrile; gradient: 0 min 2% B→0.5 min 2% B→4.5 min 90% B→9 min 90% B→9.2 min 2% B→10 min 2% B; flow rate: 0.75 ml/min; column temperature: 30° C.; UV detection: 210 nm.

### Method 8 (Analytical HPLC):

Instrument: HP 1100 with DAD detection; column: Kromasil 100 RP-18, 60 mm×2.1 mm, 3.5 μm; mobile phase A: 5 ml of HClO₄ (70% strength)/liter of water, mobile phase B: acetonitrile; gradient: 0 min 2% B→0.5 min 2% B→4.5 min 90% B→6.5 min 90% B→6.7 min 2% B→7.5 min 2% B; flow rate: 0.75 ml/min; column temperature: 30° C.; UV detection: 210 nm.

### Starting Materials and Intermediates

### Example 1A

### 1-(2-Fluorobenzyl)-N′-hydroxy-1H-pyrazolo[3,4-b]pyridine-3-carboximidamide

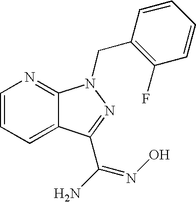

### Step a)

### 3-(Trifluoromethyl)-1H-pyrazolo[3,4-b]pyridine

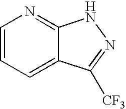

At −75° C., 2-fluoropyridine (2.00 g, 20.6 mmol) is added to a solution of freshly prepared LDA (22.7 mmol) in THF (60 ml). The solution is stirred at this temperature for 4 h. Ethyl trifluoroacetate (18.4 g, 130 mmol) is then added quickly, the internal temperature increasing to about 40° C. The mixture is once more cooled to −75° C., and hydrazine hydrate (28.9 g, 577 mmol) is then added. The reaction mixture is subsequently heated at 70° C. for 6 h. Volatile components are then removed under reduced pressure. Water (300 ml) is added to the residue, and with vigorous stirring the mixture is briefly heated to the boil. The mixture is allowed to cool to RT and filtered off with suction. The residue is taken up in ethyl acetate (300 ml), dried over sodium sulfate and clarified over activated carbon. Concentration gives 50 g (55% of theory) of the title compound as a slightly yellowish solid.

¹H-NMR (400 MHz, DMSO-d₆): δ=7.43 (dd, J=8.1, 4.4 Hz, 1H), 8.34 (d, J=8.1 Hz, 1H), 8.72 (dd, J=4.4, 1.5 Hz, 1H), 14.67 (br. s, 1H).

LC/MS (Method 2): Rₜ=1.60 min.; MS (ESIpos): m/z=188 [M+H]⁺.

### Step b)

### 1H-Pyrazolo[3,4-b]pyridine-3-carbonitrile

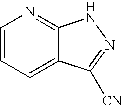

In 33% strength aqueous ammonia solution (10 ml), 3-(trifluoromethyl)-1H-pyrazolo[3,4-b]pyridine (500 mg, 2.67 mmol) is heated in the microwave at 140° C. for 10 min. The mixture is then concentrated under reduced pressure, and the residue is triturated at 70° C. with 100 ml of ethyl acetate and 20 ml of tert-butyl methyl ether. Insoluble components are filtered off with suction in the heat, and the filtrate is concentrated. Drying gives 346 mg (90% of theory) of the title compound as light-beige crystals.

¹H-NMR (400 MHz, DMSO-d₆): δ=7.47 (dd, J=8.2, 4.5 Hz, 1H), 8.46 (dd, J=8.2, 1.5 Hz, 1H), 8.73 (dd, J=4.5, 1.5 Hz, 1H), 15.02 (br. s, 1H).

LC/MS (Method 1): Rₜ=1.30 min.; MS (ESIpos): m/z=145 [M+H]⁺.

### Step c)

### 1-(2-Fluorobenzyl)-1H-pyrazolo[3,4-b]pyridine-3-carbonitrile

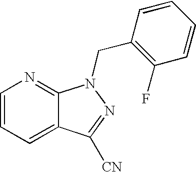

Under argon, cesium carbonate (437 mg, 1.34 mmol) is added to a solution of 1H-pyrazolo[3,4-b]pyridine-3-carbonitrile (200 mg, 88% pure, 1.22 mmol) and 2-fluorobenzyl bromide (253 mg, 1.34 mmol) in 5 ml of DMF, and the reaction mixture is stirred at RT for 16 h. For work-up, 1.5 ml of 1 N hydrochloric acid and 3 ml of DMSO are added. The resulting solution is purified directly by preparative HPLC (Method 5). This gives 250 mg (81% of theory) of the title compound.

¹H-NMR (400 MHz, DMSO-d₆): δ=5.88 (s, 2H), 7.16-7.27 (m, 2H), 7.31-7.44 (m, 2H), 7.55 (dd, 1H), 8.51 (dd, 1H), 8.81 (dd, 1H).

LC/MS (Method 1): Rₜ=2.38 min.; MS (ESIpos): m/z=253 [M+H]⁺.

### Step d)

### 1-(2-Fluorobenzyl)-N′-hydroxy-1H-pyrazolo[3,4-b]pyridine-3-carboximidamide

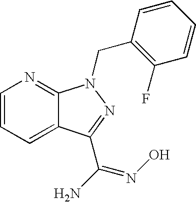

689 mg of hydroxylamine hydrochloride (9.911 mmol) are dissolved in 25 ml of DMSO, and 1.381 ml of triethylamine (1.003 g, 9.911 mmol) are added with stirring. After the addition, the mixture is stirred for 10 min and the precipitate formed is filtered off. A little at a time, 500 mg (1.982 mmol) of 1-(2-fluorobenzyl)-1H-pyrazolo[3,4-b]pyridine-3-carbonitrile are added to the filtrate, and the reaction mixture is stirred at 75° C. for 16 h. The mixture is cooled, 20 ml of water are then added and the mixture is extracted three times with ethyl acetate. The combined organic phases are washed with saturated sodium chloride solution and dried over sodium sulfate. The solvent is removed on a rotary evaporator and the residue is dried under high vacuum. This gives 718 mg of crude product which is used without further purification for subsequent reactions.

LC/MS (Method 1): Rₜ=1.82 min.; MS (ESIpos): m/z=286 [M+H]⁺.

¹H-NMR (400 MHz, CDCl₃): δ=5.33 (s, 2H), 5.80 (s, 2H), 7.00-7.10 (m, 3H), 7.18-7.28 (m, 2H), 8.48-8.50 (m, 1H), 8.57-8.59 (m, 1H).

### Example 2A

### 1-(2-Fluorobenzyl)-1H-pyrazolo[3,4-b]pyridine-3-carbohydrazide

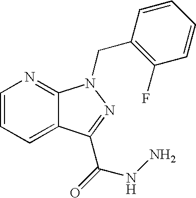

500 mg (1.671 mmol) of ethyl 1-(2-fluorobenzyl)-1H-pyrazolo[3,4-b]pyridine-3-carboxylate [prepared according to WO 03/095451, Example 2A] are dissolved in a mixture of 3 ml of methanol and 1.5 ml of THF. 1.625 ml of hydrazine monohydrate (1.672 g, 33.411 mmol) are added dropwise, and the mixture is heated at 65° C. for 4 h. After cooling, the reaction mixture is concentrated on a rotary evaporator and the residue is dried under high vacuum. This gives 540 mg of crude product which is used without further purification for subsequent reactions.

LC/MS (Method 2): Rₜ=1.47 min.; MS (ESIpos): m/z=286 [M+H]⁺.

¹H-NMR (400 MHz, DMSO-d₆): δ=4.51 (s, 2H), 5.80 (s, 2H), 7.11-7.25 (m, 3H), 7.33-7.41 (m, 2H), 8.54 (dd, J=8.1 and 1.5, 1H), 8.66 (dd, J=4.4 and 1.5, 1H), 9.72 (br. s, 1H).

### Example 3A

### 2,4-Dimethoxybenzyl isocyanate

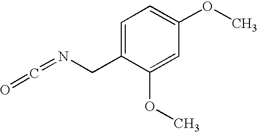

368 mg of 2,4-dimethoxybenzylamine (2.200 mmol) are dissolved in 25 ml of dichlormethane, and 15 ml of a saturated aqueous sodium bicarbonate solution are added. A little at a time, 652 mg of triphosgene (2.200 mmol) are added to the reaction mixture, which has been cooled to 0° C., and the reaction mixture is stirred at 0° C. for 30 min. The organic phase is separated off, washed with saturated sodium chloride solution and dried over sodium sulfate. The solvent is removed on a rotary evaporator and the residue (437 mg) is used without further purification for subsequent reactions [for the preparation of the title compound, cf. also B. M. Trost, D. R. Fandrick, Org. Lett. 2005, 7, 823-826].

### Example 4A

### N-(2,4-Dimethoxybenzyl)-2-{[1-(2-fluorobenzyl)-1H-pyrazolo[3,4-b]pyridin-3-yl]carbonyl}-hydrazinecarboxamide

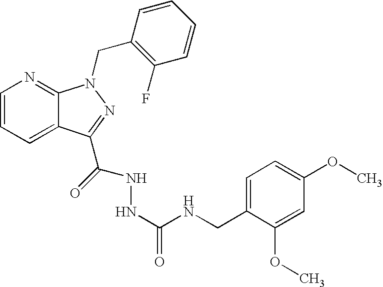

600 mg of 1-(2-fluorobenzyl)-1H-pyrazolo[3,4-b]pyridine-3-carbohydrazide (2.057 mmol, crude product from Example 2A) are dissolved in 6 ml of dichloromethane, 437 mg of 2,4-dimethoxybenzyl isocyanate (2.263 mmol, crude product from Example 3A) are added and the mixture is stirred at room temperature overnight. The precipitate formed is filtered off, washed with a little dichloromethane and dried under high vacuum. This gives 825 mg (84% of theory) of the title compound.

¹H-NMR (400 MHz, DMSO-d₆): δ=3.74 (s, 3H), 3.76 (s, 3H), 4.12 (d, J=5.62, 2H), 5.83 (s, 2H), 6.47 (dd, J=8.3 and 2.2, 1H), 6.52 (d, J=2.2, 1H), 6.66 (br. s, 1H), 7.12-7.26 (m, 4H), 7.33-7.39 (m, 1H), 7.42-7.45 (m, 1H), 7.98 (s, 1H), 8.55 (dd, J=8.1 and 1.2, 1H), 8.69 (dd, J=4.4 and 1.5, 1H), 10.14 (s, 1H).

LC/MS (Method 2): Rₜ=1.98 min.; MS (ESIpos): m/z=479 [M+H]⁺.

### Example 5A

### 4-(2,4-Dimethoxybenzyl)-5-[1-(2-fluorobenzyl)-1H-pyrazolo[3,4-b]pyridin-3-yl]-2,4-dihydro-3H-1,2,4-triazol-3-one

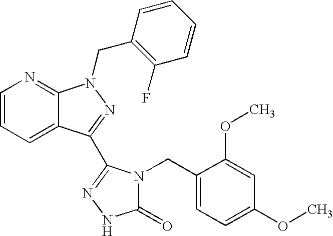

806 mg of N-(2,4-dimethoxybenzyl)-2-{[1-(2-fluorobenzyl)-1H-pyrazolo[3,4-b]pyridin-3-yl]carbonyl}hydrazinecarboxamide (Example 4A, 1.686 mmol) are suspended in 24 ml of 2% aqueous sodium hydroxide solution and heated under reflux for 16 h. After cooling, the reaction mixture is acidified to a pH of 3 using 1 N hydrochloric acid. The precipitate formed is filtered off, dried and then triturated with ethyl acetate. The solid is filtered off and dried under high vacuum. This gives 318 mg of the title compound (37% of theory, purity 91% according to LC/MS). The filtrate is extracted with 1 N aqueous sodium hydroxide solution, the organic phase is dried over sodium sulfate and concentrated on a rotary evaporator and the residue is dried under high vacuum. This gives another 288 mg of the title compound (36% of theory, purity 97% according to LC/MS).

¹H-NMR (400 MHz, DMSO-d₆): δ=3.63 (s, 3H), 3.68 (s, 3H), 5.20 (s, 2H), 5.69 (s, 2H), 6.29 (dd, J=8.5 and 2.4, 1H), 6.49 (d, J=2.4, 1H), 6.59 (d, J=8.5, 1H), 6.90-6.98 (m, 2H), 7.10-7.17 (m, 1H), 7.27-7.33 (m, 1H), 7.39-7.42 (m, 1H), 8.52 (dd, J=8.1 and 1.5, 1H), 8.66 (dd, J=4.6 and 1.5, 1H), 12.18 (br. s, 1H).

LC/MS (Method 1): Rₜ=2.33 min.; MS (ESIpos): m/z=461 [M+H].

### Example 6A

### 4-(2,4-Dimethoxybenzyl)-5-[1-(2-fluorobenzyl)-1H-pyrazolo[3,4-b]pyridin-3-yl]-2-methyl-2,4-dihydro-3H-1,2,4-triazol-3-one

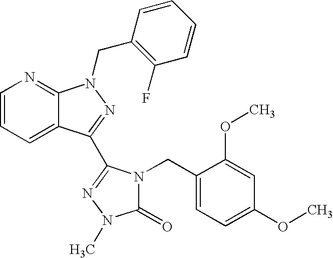

100 mg of 4-(2,4-dimethoxybenzyl)-5-[1-(2-fluorobenzyl)-1H-pyrazolo[3,4-b]pyridin-3-yl]-2,4-dihydro-3H-1,2,4-triazol-3-one (Example 5A, 0.217 mmol) are dissolved in 3 ml of DMF. 142 mg of cesium carbonate (0.434 mmol) and 62 mg of iodomethane (0.434 mmol) are added to the solution, and the reaction mixture is stirred at room temperature for 16 h. For work-up, water is added and the mixture is extracted with dichloromethane. The combined organic phases are dried over sodium sulfate, the solvent is removed on a rotary evaporator and the residue is dried under high vacuum. This gives 109 mg of the title compound (99% of theory, purity 94% according to LC/MS).

¹H-NMR (400 MHz, DMSO-d₆): δ=3.51 (s, 3H), 3.61 (s, 3H), 3.68 (s, 3H), 5.22 (s, 2H), 5.70 (s, 2H), 6.28-6.30 (m, 1H), 6.49 (d, J=2.5, 1H), 6.63 (d, J=8.6, 1H), 6.92-6.98 (m, 2H), 7.12-7.17 (m, 1H), 7.27-7.33 (m, 1H), 7.42 (dd, J=8.1 and 4.4, 1H), 8.54 (dd, J=8.1 and 1.5, 1H), 8.69 (dd, J=4.4 and 1.5, 1H).

LC/MS (Method 1): Rₜ=2.55 min.; MS (ESIpos): m/z=475 [M+H]⁺.

### Example 7A

### 4-(2,4-Dimethoxybenzyl)-2-ethyl-5-[1-(2-fluorobenzyl)-1H-pyrazolo[3,4-b]pyridin-3-yl]-2,4-dihydro-3H-1,2,4-triazol-3-one

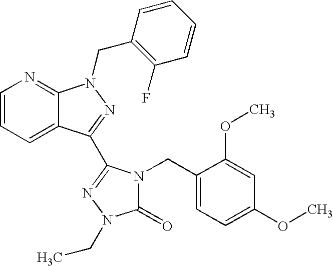

100 mg of 4-(2,4-dimethoxybenzyl)-5-[1-(2-fluorobenzyl)-1H-pyrazolo[3,4-b]pyridin-3-yl]-2,4-dihydro-3H-1,2,4-triazol-3-one (Example 5A, 0.197 mmol) are dissolved in 3 ml of DMF. 142 mg of cesium carbonate (0.434 mmol) and 68 mg of iodoethane (0.434 mmol) are added to the solution, and the reaction mixture is stirred at room temperature for 16 h. For work-up, water is added and the mixture is extracted with dichloromethane. The combined organic phases are dried over sodium sulfate, the solvent is removed on a rotary evaporator and the residue is dried under high vacuum. This gives 77 mg of the title compound (65% of theory, purity 83% according to LC/MS).

LC/MS (Method 2): Rₜ=2.51 min.; MS (ESIpos): m/z=489 [M+H]⁺.

### Example 8A

### 2-{[1-(2-Fluorobenzyl)-1H-pyrazolo[3,4-b]pyridin-3-yl]carbonyl}-N-isopropylhydrazine-carboxamide

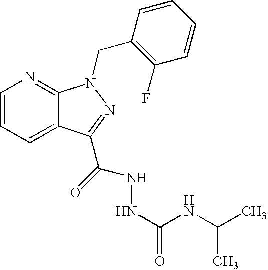

200 mg of 1-(2-fluorobenzyl)-1H-pyrazolo[3,4-b]pyridine-3-carbohydrazide (0.686 mmol, crude product from Example 2A) are dissolved in 2 ml of dichloromethane, 64 mg of isopropyl isocyanate (0.754 mmol) are added and the mixture is stirred at room temperature for 16 h. The precipitate formed is filtered off and dried under high vacuum. This gives 180 mg (70% of theory) of the title compound.

¹H-NMR (400 MHz, DMSO-d₆): δ=1.06 (d, J=6.6, 6H), 3.67-3.79 (m, 1H), 5.82 (s, 2H), 6.20 (d, J=7.6, 1H), 7.13-7.44 (m, 5H), 7.76 (s, 1H), 8.54 (d, J=8.1, 1H), 8.69 (d, J=3.9, 1H), 10.01 (s, 1H).

LC/MS (Method 1): Rₜ=1.87 min.; MS (ESIpos): m/z=371 [M+H]⁺.

### Example 9A

### 1-(2,3-Difluorobenzyl)-N′-hydroxy-1H-pyrazolo[3,4-b]pyridine-3-carboximidamide

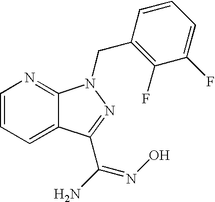

### Step a)

### 1-(2,3-Difluorobenzyl)-1H-pyrazolo[3,4-b]pyridine-3-carbonitrile

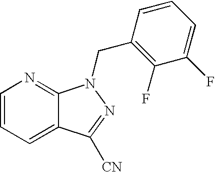

Analogously to Example 1A/Step c), 1H-pyrazolo[3,4-b]pyridine-3-carbonitrile (90 mg, 0.62 mmol) and 2,3-difluorobenzyl bromide (142 mg, 0.69 mmol) give 127 mg (75% of theory) of the title compound.

¹H-NMR (400 MHz, DMSO-d₆): δ=5.93 (s, 2H), 7.12-7.23 (m, 2H), 7.43 (dd, 1H), 7.55 (dd, 1H), 8.51 (dd, 1H), 8.81 (dd, 1H).

LC/MS (Method 2): Rₜ=2.29 min.; MS (ESIpos): m/z=271 [M+H]⁺.

### Step b)

### 1-(2,3-Difluorobenzyl)-N′-hydroxy-1H-pyrazolo[3,4-b]pyridine-3-carboximidamide

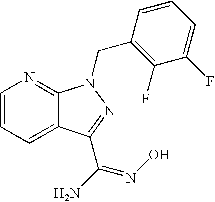

Analogously to Example 1A/Step d), 1-(2,3-difluorobenzyl)-1H-pyrazolo[3,4-b]pyridine-3-carbonitrile (58 mg, 0.22 mmol) gives 65 mg of the title compound as a crude product which is used without further purification for the subsequent reaction.

LC/MS (Method 3): Rₜ=2.15 min.; MS (ESIpos): m/z=304 [M+H]⁺.

### Example 10A

### 1-(2,5-Difluorobenzyl)-N′-hydroxy-1H-pyrazolo[3,4-b]pyridine-3-carboximidamide

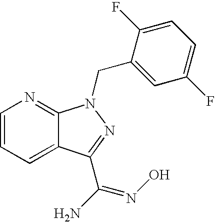

### Step a)

### 1-(2,5-Difluorobenzyl)-1H-pyrazolo[3,4-b]pyridine-3-carbonitrile

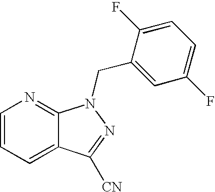

Analogously to Example 1A/Step c), 1H-pyrazolo[3,4-b]pyridine-3-carbonitrile (100 mg, 0.69 mmol) and 2,5-difluorobenzyl bromide (158 mg, 0.76 mmol) give 150 mg (80% of theory) of the title compound.

¹H-NMR (400 MHz, DMSO-d₆): δ=5.88 (s, 2H), 7.20-7.35 (m, 3H), 7.56 (dd, 1H), 8.52 (dd, 1H), 8.81 (dd, 1H).

LC/MS (Method 1): Rₜ=2.44 min.; MS (ESIpos): m/z=271 [M+H]⁺.

### Step b)

### 1-(2,5-Difluorobenzyl)-N′-hydroxy-1H-pyrazolo[3,4-b]pyridine-3-carboximidamide

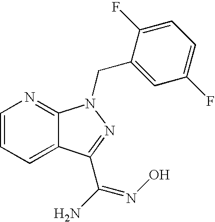

Analogously to Example 1A/Step d), 1-(2,5-difluorobenzyl)-1H-pyrazolo[3,4-b]pyridine-3-carbonitrile (75 mg, 0.28 mmol) gives 84 mg of the title compound as a crude product which is used without further purification for the subsequent reaction.

LC/MS (Method 3): Rₜ=2.12 min.; MS (ESIpos): m/z=304 [M+H]⁺.

### Example 11A

### 1-(2,6-Difluorobenzyl)-N′-hydroxy-1H-pyrazolo[3,4-b]pyridine-3-carboximidamide

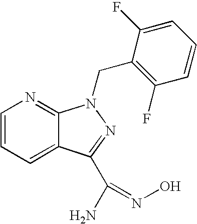

### Step a)

### 1-(2,6-Difluorobenzyl)-1H-pyrazolo[3,4-b]pyridine-3-carbonitrile

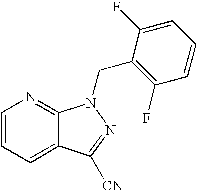

Analogously to Example 1A/Step c), 1H-pyrazolo[3,4-b]pyridine-3-carbonitrile (100 mg, 0.69 mmol) and 2,6-difluorobenzyl bromide (158 mg, 0.63 mmol) give 161 mg (86% of theory) of the title compound.

¹H-NMR (400 MHz, DMSO-d₆): δ=5.88 (s, 2H), 7.12-7.20 (m, 2H), 7.46-7.58 (m, 2H), 8.49 (dd, 1H), 8.82 (dd, 1H).

LC/MS (Method 2): Rₜ=2.20 min.; MS (ESIpos): m/z=271 [M+H]⁺.

### Step b)

### 1-(2,6-Difluorobenzyl)-N′-hydroxy-1H-pyrazolo[3,4-b]pyridine-3-carboximidamide

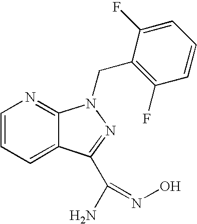

Analogously to Example 1A/Step d), 1-(2,6-difluorobenzyl)-1H-pyrazolo[3,4-b]pyridine-3-carbonitrile (90 mg, 0.33 mmol) gives 100 mg of the title compound as a crude product which is used without further purification for the subsequent reaction.

LC/MS (Method 1): Rₜ=1.84 min.; MS (ESIpos): m/z=304 [M+H]⁺.

### Example 12A

### 1-[(5-Chloro-2-thienyl)methyl]-N′-hydroxy-1H-pyrazolo[3,4-b]pyridine-3-carboximidamide

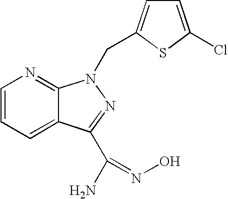

### Step a)

### 1-[(5-Chloro-2-thienyl)methyl]-1H-pyrazolo[3,4-b]pyridine-3-carbonitrile

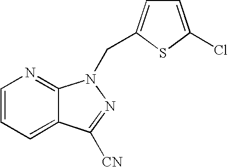

Analogously to Example 1A/Step c), 1H-pyrazolo[3,4-b]pyridine-3-carbonitrile (100 mg, 90% pure, 0.62 mmol) and 2-chloro-5-(chloromethyl)thiophene (125 mg, 0.75 mmol) give 130 mg (74% of theory) of the title compound.

¹H-NMR (400 MHz, DMSO-d₆): δ=5.97 (s, 2H), 7.01 (d, 1H), 7.12 (d, 1H), 7.56 (dd, 1H), 8.51 (dd, 1H), 8.82 (dd, 1H).

### Step b)

### 1-[(5-Chloro-2-thienyl)methyl]-N′-hydroxy-1H-pyrazolo[3,4-b]pyridine-3-carboximidamide

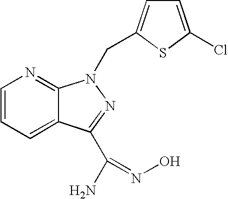

Analogously to Example 1A/Step d), 1-[(5-chloro-2-thienyl)methyl]-1H-pyrazolo[3,4-b]pyridine-3-carbonitrile (57 mg, 0.21 mmol) gives 60 mg of the title compound as a crude product which is used without further purification for the subsequent reaction.

LC/MS (Method 1): Rₜ=2.00 min.; MS (ESIpos): m/z=308 [M+H]⁺.

### Example 13A

### 4-(2,4-Dimethoxybenzyl)-5-[1-(2-fluorobenzyl)-1H-pyrazolo[3,4-b]pyridin-3-yl]-2-propyl-2,4-dihydro-3H-1,2,4-triazol-3-one

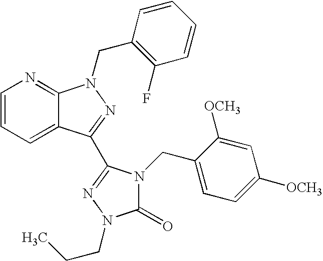

4-(2,4-Dimethoxybenzyl)-5-[1-(2-fluorobenzyl)-1H-pyrazolo[3,4-b]pyridin-3-yl]-2,4-dihydro-3H-1,2,4-triazol-3-one from Example 5A (300 mg, 0.65 mmol) is suspended in DMF (2.5 ml), and sodium hydride (60% in mineral oil, 78 mg, 1.96 mmol) is added. The mixture is stirred at RT for 2 h, and 1-iodopropane (421 mg, 2.48 mmol) is then added. After 20 h of stirring at RT, the mixture is diluted with water and extracted with ethyl acetate. The organic phase is dried over sodium sulfate and concentrated. The residue is purified by preparative HPLC, giving 241 mg (74% of theory) of the title compound as a colorless oil.

¹H-NMR (400 MHz, DMSO-d₆): δ=0.92 (t, J=7.5 Hz, 3H), 1.79 (tq, J=7.5, 6.9 Hz, 2H), 3.63 (s, 3H), 3.68 (s, 3H), 3.82 (t, J=6.9 Hz, 2H), 5.23 (s, 2H), 5.70 (s, 2H), 6.27 (dd, J=8.3, 2.2 Hz, 1H), 6.49 (d, J=2.2 Hz, 1H), 6.59 (d, J=8.3 Hz, 1H), 6.91-6.99 (m, 2H), 7.12-7.18 (m, 1H), 7.27-7.34 (m, 1H), 7.42 (dd, J=8.1, 4.4 Hz, 1H), 8.53 (dd, J=8.1, 1.5 Hz, 1H), 8.69 (dd, J=4.4, 1.5 Hz, 1H).

LC/MS (Method 2): Rₜ=2.69 min.; MS (ESIpos): m/z=503 [M+H]⁺.

### Example 14A

### 4-(2,4-Dimethoxybenzyl)-5-[1-(2-fluorobenzyl)-1H-pyrazolo[3,4-b]pyridin-3-yl]-2-isobutyl-2,4-dihydro-3H-1,2,4-triazol-3-one

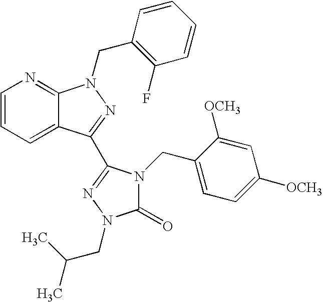

Analogously to Example 13A, the title compound is synthesized from 4-(2,4-dimethoxybenzyl)-5-[1-(2-fluorobenzyl)-1H-pyrazolo[3,4-b]pyridin-3-yl]-2,4-dihydro-3H-1,2,4-triazol-3-one (Example 5A; 300 mg, 0.65 mmol) and 1-iodo-2-methylpropane (456 mg, 2.48 mmol). The crude product is purified by preparative HPLC, giving 241 mg (72% of theory) of the title compound as white crystals.

¹H-NMR (400 MHz, DMSO-d₆): δ=0.94 (d, J=6.8 Hz, 6H), 2.12-2.25 (m, 1H), 3.64 (s, 3H), 3.68 (s, 3H), 3.68 (d, J=6.8 Hz, 2H), 5.23 (s, 2H), 5.70 (s, 2H), 6.28 (dd, J=8.3, 2.2 Hz, 1H), 6.49 (d, J=2.2 Hz, 1H), 6.58 (d, J=8.3 Hz, 1H), 6.90-6.99 (m, 2H), 7.12-7.18 (m, 1H), 7.27-7.34 (m, 1H), 7.42 (dd, J=8.1, 4.4 Hz, 1H), 8.52 (dd, J=8.1, 1.5 Hz, 1H), 8.69 (dd, J=4.4, 1.5 Hz, 1H).

LC/MS (Method 2): Rₜ=2.84 min.; MS (ESIpos): m/z=517 [M+H]⁺.

### Example 15A

### 4-(2,4-Dimethoxybenzyl)-5-[1-(2-fluorobenzyl)-1H-pyrazolo[3,4-b]pyridin-3-yl]-2-isopropyl-2,4-dihydro-3H-1,2,4-triazol-3-one

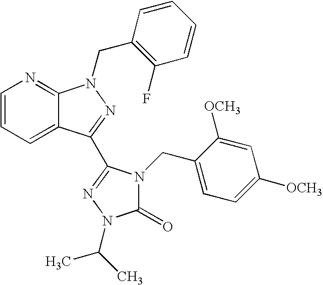

Analogously to Example 13A, the title compound is synthesized from 4-(2,4-dimethoxybenzyl)-5-[1-(2-fluorobenzyl)-1H-pyrazolo[3,4-b]pyridin-3-yl]-2,4-dihydro-3H-1,2,4-triazol-3-one (Example 5A; 300 mg, 0.65 mmol) and 2-iodopropane (421 mg, 2.48 mmol). The crude product is purified by preparative HPLC, giving 249 mg (74% of theory) of the title compound as a colorless oil.

¹H-NMR (400 MHz, DMSO-d₆): δ=1.41 (d, J=6.6 Hz, 6H), 3.63 (s, 3H), 3.68 (s, 3H), 4.47 (sept, J=6.6 Hz, 1H), 5.22 (s, 2H), 5.70 (s, 2H), 6.29 (dd, J=8.3, 2.2 Hz, 1H), 6.49 (d, J=2.2 Hz, 1H), 6.58 (d, J=8.3 Hz, 1H), 6.89-6.99 (m, 2H), 7.12-7.18 (m, 1H), 7.27-7.34 (m, 1H), 7.43 (dd, J=8.1, 4.4 Hz, 1H), 8.56 (dd, J=8.1, 1.5 Hz, 1H), 8.69 (dd, J=4.4, 1.5 Hz, 1H).

LC/MS (Method 2): Rₜ=2.72 min.; MS (ESIpos): m/z=503 [M+H]⁺.

### Example 16A

### 4-(2,4-Dimethoxybenzyl)-5-[1-(2-fluorobenzyl)-1H-pyrazolo[3,4-b]pyridin-3-yl]-2-(2-fluoroethyl)-2,4-dihydro-3H-1,2,4-triazol-3-one

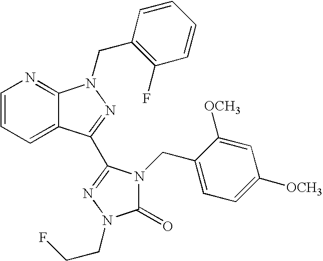

Analogously to Example 13A, the title compound is synthesized from 4-(2,4-dimethoxybenzyl)-5-[1-(2-fluorobenzyl)-1H-pyrazolo[3,4-b]pyridin-3-yl]-2,4-dihydro-3H-1,2,4-triazol-3-one (Example 5A; 300 mg, 0.65 mmol) and 1-bromo-2-fluoroethane (314 mg, 2.48 mmol). The crude product is synthesized by preparative HPLC, giving 250 mg (76% of theory) of the title compound as white crystals.

¹H-NMR (400 MHz, DMSO-d₆): δ=3.61 (s, 3H), 3.68 (s, 3H), 4.18 (dt, J=26.9, 4.8 Hz, 2H), 4.81 (dt, J=47.2, 4.8 Hz, 2H), 5.24 (s, 2H), 5.71 (s, 2H), 6.29 (dd, J=8.3, 2.2 Hz, 1H), 6.49 (d, J=2.2 Hz, 1H), 6.63 (d, J=8.3 Hz, 1H), 6.92-7.00 (m, 2H), 7.13-7.19 (m, 1H), 7.28-7.35 (m, 1H), 7.43 (dd, J=8.1, 4.4 Hz, 1H), 8.56 (dd, J=8.1, 1.5 Hz, 1H), 8.70 (dd, J=4.4, 1.5 Hz, 1H).

LC/MS (Method 2): Rₜ=2.45 min.; MS (ESIpos): m/z=507 [M+H]⁺.

### Example 17A

### 2-(2,2-Difluoroethyl)-4-(2,4-dimethoxybenzyl)-5-[1-(2-fluorobenzyl)-1H-pyrazolo[3,4-b]pyridin-3-yl]-2,4-dihydro-3H-1,2,4-triazol-3-one

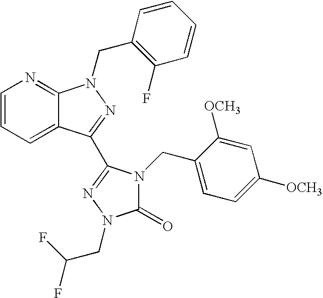

Analogously to Example 13A, the title compound is synthesized from 4-(2,4-dimethoxybenzyl)-5-[1-(2-fluorobenzyl)-1H-pyrazolo[3,4-b]pyridin-3-yl]-2,4-dihydro-3H-1,2,4-triazol-3-one (Example 5A; 300 mg, 0.65 mmol) and 2-bromo-1,1-difluoroethane (358 mg, 2.48 mmol). The crude product is purified by preparative HPLC, giving 273 mg (76% of theory) of the title compound as white crystals.

¹H-NMR (400 MHz, DMSO-d₆): δ=3.60 (s, 3H), 3.68 (s, 3H), 4.33 (dt, J=15.0, 3.5 Hz, 2H), 5.24 (s, 2H), 5.72 (s, 2H), 6.29 (dd, J=8.3, 2.2 Hz, 1H), 6.45 (tt, J=54.8, 3.5 Hz, 1H), 6.48 (d, J=2.2 Hz, 1H), 6.65 (d, J=8.3 Hz, 1H), 6.93-7.01 (m, 2H), 7.13-7.19 (m, 1H), 7.28-7.35 (m, 1H), 7.44 (dd, J=8.1, 4.4 Hz, 1H), 8.54 (dd, J=8.1, 1.5 Hz, 1H), 8.70 (dd, J=4.4, 1.5 Hz, 1H).

LC/MS (Method 2): Rₜ=2.55 min.; MS (ESIpos): m/z=525 [M+H]⁺.

### Example 18A

### 4-(2,4-Dimethoxybenzyl)-5-[1-(2-fluorobenzyl)-1H-pyrazolo[3,4-b]pyridin-3-yl]-2-(2,2,2-trifluoroethyl)-2,4-dihydro-3H-1,2,4-triazol-3-one

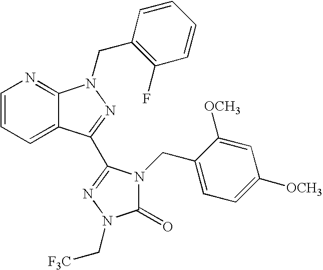

4-(2,4-Dimethoxybenzyl)-5-[1-(2-fluorobenzyl)-1H-pyrazolo[3,4-b]pyridin-3-yl]-2,4-dihydro-3H-1,2,4-triazol-3-one (Example 5A; 100 mg, 0.22 mmol) is suspended in DMF (3.0 ml), and cesium carbonate (156 mg, 0.48 mmol) and 2,2,2-trifluoroethyl trichloromethylsulfonate (122 mg, 0.43 mmol) are added. After 20 h of stirring at RT, the mixture is warmed at 60° C. for another 3 h. The mixture is then diluted with water and extracted with dichloromethane. The organic phase is dried over sodium sulfate and concentrated. The crude product is purified by preparative HPLC, giving 119 mg (99% of theory) of the title compound as a beige solid.

¹H-NMR (400 MHz, DMSO-d₆): δ=3.58 (s, 3H), 3.68 (s, 3H), 4.81 (q, J=9.1 Hz, 2H), 5.24 (s, 2H), 5.72 (s, 2H), 6.28 (dd, J=8.3, 2.2 Hz, 1H), 6.48 (d, J=2.2 Hz, 1H), 6.66 (d, J=8.3 Hz, 1H), 6.96-7.02 (m, 2H), 7.16 (dd, J=10.3, 8.3 Hz, 1H), 7.29-7.35 (m, 1H), 7.46 (dd, J=8.1, 4.4 Hz, 1H), 8.47 (dd, J=8.1, 1.5 Hz, 1H), 8.71 (dd, J=4.4, 1.5 Hz, 1H).

LC/MS (Method 1): Rₜ=2.87 min.; MS (ESIpos): m/z=543 [M+H]⁺.

### Example 19A

### 4-(2,4-Dimethoxybenzyl)-5-[1-(2-fluorobenzyl)-1H-pyrazolo[3,4-b]pyridin-3-yl]-2-(2-hydroxyethyl)-2,4-dihydro-3H-1,2,4-triazol-3-one

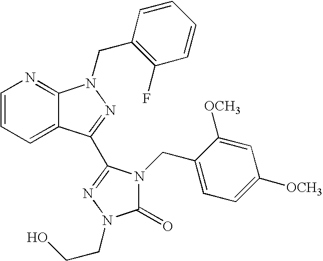

4-(2,4-Dimethoxybenzyl)-5-[1-(2-fluorobenzyl)-1H-pyrazolo[3,4-b]pyridin-3-yl]-2,4-dihydro-3H-1,2,4-triazol-3-one (Example 5A; 50% pure, 500 mg, 0.54 mmol) is suspended in DMF (2.0 ml), and sodium hydride (60% in mineral oil, 65 mg, 1.63 mmol) is added. The mixture is stirred at RT for 2 h, and 2-iodoethanol (354 mg, 2.06 mmol) is then added. After 20 h of stirring at RT, the mixture is diluted with water and extracted with ethyl acetate. The organic phase is dried over sodium sulfate and concentrated. The crude product is purified by preparative HPLC, giving 199 mg (73% of theory) of the title compound as a light-beige solid.

¹H-NMR (400 MHz, DMSO-d₆): δ=3.63 (s, 3H), 3.68 (s, 3H), 3.75-3.80 (m, 2H), 3.90 (t, J=5.8 Hz, 2H), 4.90 (br. s, 1H), 5.23 (s, 2H), 5.70 (s, 2H), 6.29 (dd, J=8.3, 2.2 Hz, 1H), 6.49 (d, J=2.2 Hz, 1H), 6.64 (d, J=8.3 Hz, 1H), 6.88-6.99 (m, 2H), 7.15 (dd, J=10.3, 8.3 Hz, 1H), 7.27-7.34 (m, 1H), 7.42 (dd, J=8.1, 4.4 Hz, 1H), 8.55 (dd, J=8.1, 4.4 Hz, 1H), 8.55 (dd, J=8.1, 1.5 Hz, 1H), 8.69 (dd, J=4.4, 1.5 Hz, 1H).

LC/MS (Method 1): Rₜ=2.29 min; MS (ESIpos): m/z=505 [M+H]⁺.

### Example 20A

### 4-(2,4-Dimethoxybenzyl)-5-[1-(2-fluorobenzyl)-1H-pyrazolo[3,4-b]pyridin-3-yl]-2-(3-hydroxy-propyl)-2,4-dihydro-3H-1,2,4-triazol-3-one

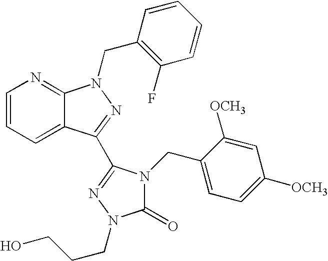

4-(2,4-Dimethoxybenzyl)-5-[1-(2-fluorobenzyl)-1H-pyrazolo[3,4-b]pyridin-3-yl]-2,4-dihydro-3H-1,2,4-triazol-3-one (Example 5A; 50% pure, 500 mg, 0.54 mmol) is suspended in DMF (2.5 ml), and sodium hydride (60% in mineral oil, 65 mg, 1.63 mmol) is added. The mixture is stirred at RT for 2 h, and 3-iodo-1-propanol (388 mg, 2.06 mmol) is then added. After 20 h of stirring at RT, the mixture is diluted with water and extracted with ethyl acetate. The organic phase is dried over sodium sulfate and concentrated. The crude product is purified by preparative HPLC, giving 163 mg (55% of theory) of the title compound as a light-beige solid.

¹H-NMR (400 MHz, DMSO-d₆): δ=1.93 (tt, J=7.1, 6.2 Hz, 2H), 3.51 (dt, J=6.2, 5.0 Hz, 2H), 3.63 (s, 3H), 3.68 (s, 3H), 3.92 (t, J=7.1 Hz, 2H), 4.59 (t, J=5.0 Hz, 1H), 5.22 (s, 2H), 5.70 (s, 2H), 6.29 (dd, J=8.3, 2.2 Hz, 1H), 6.49 (d, J=2.2 Hz, 1H), 6.60 (d, J=8.3 Hz, 1H), 6.90-6.99 (m, 2H), 7.15 (dd, J=10.3, 8.3 Hz, 1H), 7.27-7.34 (m, 1H), 7.42 (dd, J=8.1, 4.4 Hz, 1H), 8.54 (dd, J=8.1, 1.5 Hz, 1H), 8.69 (dd, J=4.4, 1.5 Hz, 1H).

LC/MS (Method 1): Rₜ=2.36 min.; MS (ESIpos): m/z=519 [M+H]⁺.

### Example 21A

### 4-(2,4-Dimethoxybenzyl)-5-[1-(2-fluorobenzyl)-1H-pyrazolo[3,4-b]pyridin-3-yl]-2-(3-morpholin-4-ylpropyl)-2,4-dihydro-3H-1,2,4-triazol-3-one

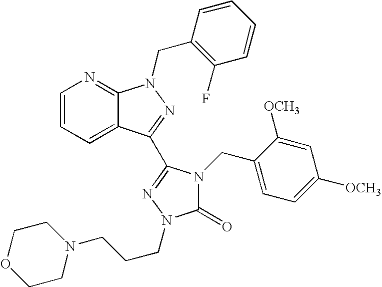

4-(2,4-Dimethoxybenzyl)-5-[1-(2-fluorobenzyl)-1H-pyrazolo[3,4-b]pyridin-3-yl]-2,4-dihydro-3H-1,2,4-triazol-3-one (Example 5A; 250 mg, 0.54 mmol) is suspended in DMF (2 ml), and sodium hydride (60% in mineral oil, 65 mg, 1.63 mmol) is added. The mixture is stirred at RT for 2 h, and 4-(3-chloropropyl)morpholine (338 mg, 2.06 mmol) and tetra-n-butylammonium iodide (762 mg, 2.06 mmol) are then added. The reaction mixture is stirred at RT for 20 h. The mixture is then stirred into ice-water and extracted with ethyl acetate. After concentration of the organic phase, the residue is purified by preparative HPLC. This gives 300 mg (94% of theory) of the title compound as a white solid.

¹H-NMR (400 MHz, DMSO-d₆): δ=1.92 (quint, J=6.8 Hz, 2H), 2.30-2.37 (m, 6H), 3.53 (t, J=4.5 Hz, 4H), 3.61 (s, 3H), 3.68 (s, 3H), 3.90 (t, J=6.8 Hz, 2H), 5.23 (s, 2H), 5.70 (s, 2H), 6.28 (dd, J=8.4, 2.4 Hz, 1H), 6.49 (d, J=2.4 Hz, 1H), 6.61 (d, J=8.4 Hz, 1H), 6.91-6.99 (m, 2H), 7.15 (dd, J=10.3, 8.3 Hz, 1H), 7.27-7.34 (m, 1H), 7.42 (dd, J=8.1, 4.7 Hz, 1H), 8.53 (dd, J=8.1, 1.4 Hz, 1H), 8.69 (dd, J=4.7, 1.4 Hz, 1H).

LC/MS (Method 2): Rₜ=1.57 min.; MS (ESIpos): m/z=588 [M+H]⁺.

### Example 22A

### 3-{4-(2,4-Dimethoxybenzyl)-3-[1-(2-fluorobenzyl)-1H-pyrazolo[3,4-b]pyridin-3-yl]-5-oxo-4,5-dihydro-1H-1,2,4-triazol-1-yl}propanenitrile

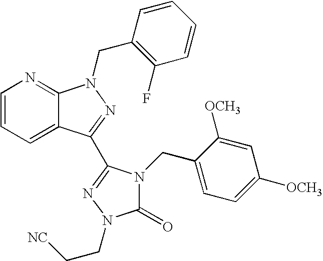

4-(2,4-Dimethoxybenzyl)-5-[1-(2-fluorobenzyl)-1H-pyrazolo[3,4-b]pyridin-3-yl]-2,4-dihydro-3H-1,2,4-triazol-3-one (Example 5A; 194 mg, 0.42 mmol) is stirred together with potassium hydroxide (4.2 mg, 0.075 mmol) and acrylonitrile (2.0 ml) at RT for two days. Ethanol (4 ml) is then added, and the mixture is heated at reflux. The mixture is filtered off with suction from the undissolved residue. In the filtrate, a further product fraction precipitates out. This fraction is filtered off with suction, giving a total of 193 mg (89% of theory) of the title compound.

¹H-NMR (400 MHz, CDCl₃): δ=3.09 (t, J=6.2 Hz, 2H), 3.63 (s, 3H), 3.68 (s, 3H), 4.14 (t, J=6.2 Hz, 2H), 5.24 (s, 2H), 5.71 (s, 2H), 6.26 (dd, J=8.6, 2.2 Hz, 1H), 6.49 (d, J=2.2 Hz, 1H), 6.65 (d, J=8.3 Hz, 1H), 6.91-7.00 (m, 2H), 7.13-7.19 (m, 1H), 7.27-7.35 (m, 1H), 7.43 (dd, J=8.1, 4.4 Hz, 1H), 8.62 (dd, J=8.1, 1.2 Hz, 1H), 8.70 (dd, J=4.4, 1.2 Hz, 1H).

LC/MS (Method 1): Rₜ=2.50 min.; MS (ESIpos): m/z=514 [M+H]⁺.

### Example 23A

### 1-(3-Fluorobenzyl)-N′-hydroxy-1H-pyrazolo[3,4-b]pyridine-3-carboximidamide

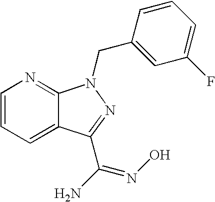

### Step a)

### 1-(3-Fluorobenzyl)-1H-pyrazolo[3,4-b]pyridine-3-carbonitrile

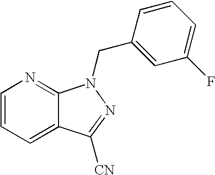

Analogously to Example 1A/Step c), 1H-pyrazolo[3,4-b]pyridine-3-carbonitrile (90 mg, 0.62 mmol) and 3-fluorobenzyl bromide (130 mg, 0.69 mmol) give 115 mg (73% of theory) of the title compound.

¹H-NMR (400 MHz, DMSO-d₆): δ=5.88 (s, 2H), 7.10-7.19 (m, 3H), 7.40 (q, 1H), 7.54 (dd, 1H), 8.51 (dd, 1H), 8.81 (dd, 1H).

LC/MS (Method 1): Rₜ=2.44 min.; MS (ESIpos): m/z=253 [M+H]⁺.

### Step b)

### 1-(3-Fluorobenzyl)-N′-hydroxy-1H-pyrazolo[3,4-b]pyridine-3-carboximidamide

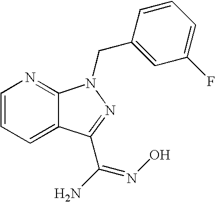

Analogously to Example 1A/Step d), 1-(3-fluorobenzyl)-1H-pyrazolo[3,4-b]pyridine-3-carbonitrile (57 mg, 0.23 mmol) gives 64 mg of the title compound as a crude product which is used without further purification for the subsequent reaction.

LC/MS (Method 3): Rₜ=2.09 min.; MS (ESIpos): m/z=286 [M+H]⁺.

### Example 24A

### 1-(2,4-Difluorobenzyl)-N′-hydroxy-1H-pyrazolo[3,4-b]pyridine-3-carboximidamide

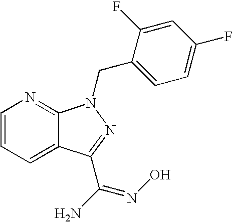

### Step a)

### 1-(2,4-Difluorobenzyl)-1H-pyrazolo[3,4-b]pyridine-3-carbonitrile

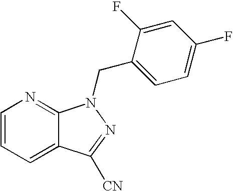

Analogously to Example 1A/Step c), 1H-pyrazolo[3,4-b]pyridine-3-carbonitrile (90 mg, 0.62 mmol) and 2,4-difluorobenzyl bromide (142 mg, 0.69 mmol) give 124 mg (73% of theory) of the title compound.

¹H-NMR (400 MHz, DMSO-d₆): δ=5.85 (s, 2H), 7.10 (dt, 1H), 7.29 (dt, 1H), 7.47 (q, 1H), 7.54 (dd, 1H), 8.50 (dd, 1H), 8.80 (dd, 1H).

LC/MS (Method 2): Rₜ=2.29 min.; MS (ESIpos): m/z=271 [M+H]⁺.

### Step b)

### 1-(2,4-Difluorobenzyl)-N′-hydroxy-1H-pyrazolo[3,4-b]pyridine-3-carboximidamide

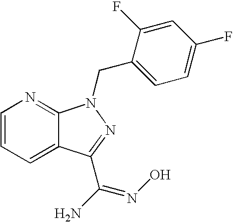

Analogously to Example 1A/Step d), 1-(2,4-difluorobenzyl)-1H-pyrazolo[3,4-b]pyridine-3-carbonitrile (68 mg, 0.25 mmol) gives 76 mg of the title compound as a crude product which is used without further purification for the subsequent reaction.

LC/MS (Method 3): Rₜ=2.15 min.; MS (ESIpos): m/z=304 [M+H]⁺.

### Example 25A

### 1-(5-Chloro-2-fluorobenzyl)-N′-hydroxy-1H-pyrazolo[3,4-b]pyridine-3-carboximidamide

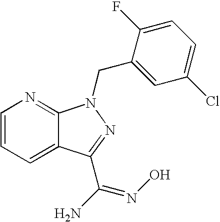

### Step a)

### 1-(5-Chloro-2-fluorobenzyl)-1H-pyrazolo[3,4-b]pyridine-3-carbonitrile

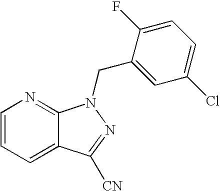

Analogously to Example 1A/Step c), 1H-pyrazolo[3,4-b]pyridine-3-carbonitrile (110 mg, 0.76 mmol) and 5-chloro-2-fluorobenzyl bromide (188 mg, 0.84 mmol) give 154 mg (70% of theory) of the title compound.

¹H-NMR (400 MHz, DMSO-d₆): δ=5.87 (s, 2H), 7.31 (dd, 1H), 7.46-7.51 (m, 2H), 7.55 (dd, 1H), 8.51 (dd, 1H), 8.81 (dd, 1H).

LC/MS (Method 1): Rₜ=2.59 min.; MS (ESIpos): m/z=287 [M+H]⁺.

### Step b)

### 1-(5-Chloro-2-fluorobenzyl)-N′-hydroxy-1H-pyrazolo[3,4-b]pyridine-3-carboximidamide

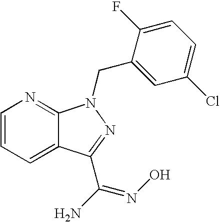

Analogously to Example 1A/Step d), 1-(5-chloro-2-fluorobenzyl)-1H-pyrazolo[3,4-b]pyridine-3-carbonitrile (64 mg, 0.22 mmol) gives 71 mg of the title compound as a crude product which is used without further purification for the subsequent reaction.

LC/MS (Method 4): Rₜ=2.25 min.; MS (ESIpos): m/z=320 [M+H]⁺.

### Example 26A

### 1-(2-Fluoro-3-methylbenzyl)-N′-hydroxy-1H-pyrazolo[3,4-b]pyridine-3-carboximidamide

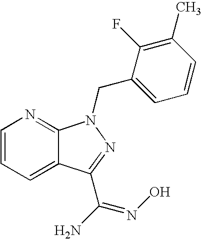

### Step a)

### 1-(2-Fluoro-3-methylbenzyl)-1H-pyrazolo[3,4-b]pyridine-3-carbonitrile

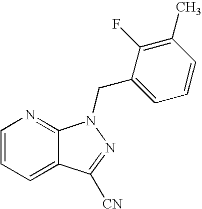

Analogously to Example 1A/Step c), 1H-pyrazolo[3,4-b]pyridine-3-carbonitrile (90 mg, 0.62 mmol) and 2-fluoro-3-methylbenzyl bromide (139 mg, 0.69 mmol) give 120 mg (72% of theory) of the title compound.

¹H-NMR (400 MHz, DMSO-d₆): δ=2.21 (s, 3H), 5.85 (s, 2H), 7.05 (t, 1H), 7.12 (t, 1H), 7.26 (t, 1H), 7.53 (dd, 1H), 8.50 (dd, 1H), 8.80 (dd, 1H).

LC/MS (Method 2): Rₜ=2.43 min.; MS (ESIpos): m/z=267 [M+H]⁺.

### Step b)

### 1-(2-Fluoro-3-methylbenzyl)-N′-hydroxy-1H-pyrazolo[3,4-b]pyridine-3-carboximidamide

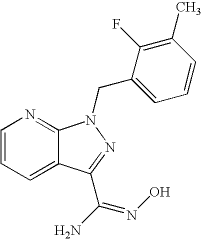

Analogously to Example 1A/Step d), 1-(2-fluoro-3-methylbenzyl)-1H-pyrazolo[3,4-b]pyridine-3-carbonitrile (51 mg, 0.19 mmol) gives 57 mg of the title compound as a crude product which is used without further purification for the subsequent reaction.

LC/MS (Method 4): Rₜ=2.25 min.; MS (ESIpos): m/z=300 [M+H]⁺.

### Example 27A

### 1-(Cycloheptylmethyl)-N′-hydroxy-1H-pyrazolo[3,4-b]pyridine-3-carboximidamide

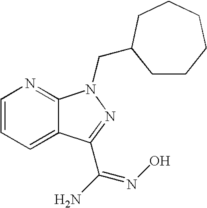

### Step a)

### 1-(Cycloheptylmethyl)-1H-pyrazolo[3,4-b]pyridine-3-carbonitrile

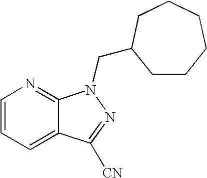

290 mg of 1H-pyrazolo[3,4-b]pyridine-3-carbonitrile (2.012 mmol; Example 1A/Step b) are dissolved in 5 ml of DMF, 419 mg of cycloheptylmethyl methanesulfonate (2.012 mmol) and 656 mg of cesium carbonate (2.012 mmol) are added and the mixture is stirred at room temperature for 16 h. Subsequently, another 200 mg of cycloheptylmethyl methanesulfonate (0.969 mmol) and 320 mg of cesium carbonate (0.982 mmol) are added, and the reaction mixture is stirred at room temperature for a further 2 days. Another 180 mg of cycloheptylmethyl methanesulfonate (0.872 mmol) and 282 mg of cesium carbonate (0.865 mmol) are added, and the reaction mixture is stirred at room temperature for another 2 days. Water is then added, and the reaction mixture is extracted three times with dichloromethane. The combined organic phases are washed with saturated sodium chloride solution, dried over sodium sulfate and concentrated on a rotary evaporator. The residue is purified chromatographically on silica gel (mobile phase: cyclohexane/ethyl acetate 5:1→2:1). This gives 433 mg (85% of theory) of the target compound.

¹H-NMR (400 MHz, DMSO-d₆): δ=1.17-1.73 (m, 12H), 2.22-2.28 (m, 1H), 3.98 (d, J=6.4, 2H), 7.51 (dd, J=8.1, 4.4, 1H), 8.48 (dd, J=8.1, 1.5, 1H), 8.76-8.77 (m, 1H).

LC/MS (Method 2): Rₜ=2.82 min.; MS (ESIpos): m/z=255 [M+H]⁺.

### Step b)

### 1-(Cycloheptylmethyl)-N′-hydroxy-1H-pyrazolo[3,4-b]pyridine-3-carboximidamide

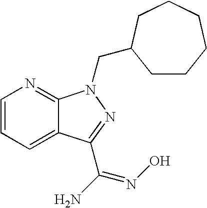

383 mg of hydroxylamine hydrochloride (5.504 mmol) are dissolved in 25 ml of dimethyl sulfoxide, and 0.767 ml of triethylamine (557 mg, 5.504 mmol) is added with stirring. After the addition has ended, the mixture is stirred for 10 min and the precipitate formed is filtered off. A little at a time, 280 mg of 1-(cycloheptylmethyl)-1H-pyrazolo[3,4-b]pyridine-3-carbonitrile (1.101 mmol) are added to the filtrate, and the reaction mixture is stirred at 75° C. for 16 h. The mixture is cooled, 25 ml of water are then added and the mixture is extracted three times with ethyl acetate. The combined organic phases are washed with saturated sodium chloride solution and dried over sodium sulfate. The solvent is removed on a rotary evaporator, and the residue is dried under high vacuum. This gives 387 mg of crude product which is used without further purification for the subsequent reactions.

LC/MS (Method 2): Rₜ=2.13 min.; MS (ESIpos): m/z=288 [M+H]⁺.

### Example 28A

### 4-Amino-1-(2-fluorobenzyl)-N′-hydroxy-1H-pyrazolo[3,4-d]pyrimidine-3-carboximidamide

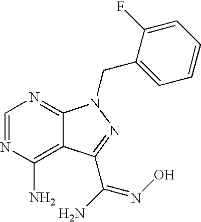

### Step a)

### 5-Amino-3,4-dicyano-1-(2-fluorobenzyl)pyrazole

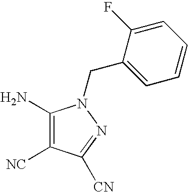

1.0 g of 2-fluorobenzylhydrazine (7.14 mmol) in 20 ml of ethanol is cooled to 0° C., and 1.005 g of tetracyanoethylene (7.85 mmol) are added. The mixture is stirred at 0° C. for 1 h and then heated at reflux for 1 h. After cooling, the reaction mixture is purified by preparative HPLC (Method 5). This gives 772 mg (45% of theory) of the title compound.

LC/MS (Method 1): Rₜ=2.01 min.; MS (ESIneg): m/z=240 [M−H]⁻

¹H-NMR (400 MHz, DMSO-d₆): δ=5.32 (s, 2H), 7.08 (t, 1H), 7.16-7.29 (m, 2H), 7.40 (m, 1H), 7.47 (s, 2H).

### Step b)

### 4-Amino-1-(2-fluorobenzyl)-1H-pyrazolo[3,4-d]pyrimidine-3-carbonitrile

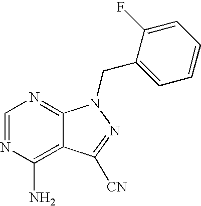

772 mg of 5-amino-3,4-dicyano-1-(2-fluorobenzyl)pyrazole (3.20 mmol) are dissolved in 15 ml of triethyl orthoformate, and the mixture is heated under reflux overnight. Excess triethyl orthoformate is removed on a rotary evaporator, and the residue is stirred at RT with 10 ml of an NH₃ solution (7 N in methanol) for 1 h. The volatile components are removed on a rotary evaporator, and the residue is triturated with diethyl ether. The solid is filtered off with suction and dried under high vacuum. This gives 650 mg (56% of theory) of the title compound in a purity of 74% (according to LC/MS). The product is used without further purification for the subsequent step.

LC/MS (Method 2): Rₜ=1.72 min.; MS (ESIneg): m/z=267 [M−H]⁻

¹H-NMR (400 MHz, DMSO-d₆): δ=5.67 (s, 2H), 7.08-7.31 (m, 3H), 7.39 (m, 1H), 7.5-8.2 (br, 2H), 8.37 (s, 1H).

### Step e)

### 4-Amino-1-(2-fluorobenzyl)-N′-hydroxy-1H-pyrazolo[3,4-d]pyrimidine-3-carboximidamide

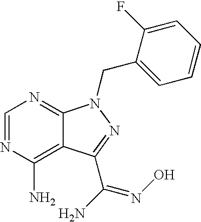

130 mg of hydroxylamine hydrochloride (1.86 mmol) are dissolved in 5 ml of DMSO, and 260 μl of triethylamine (1.86 mmol) are added. After 10 min, the precipitate formed is filtered off with suction. 100 mg of 4-amino-1-(2-fluorobenzyl)-1H-pyrazolo[3,4-d]pyrimidine-3-carbonitrile (0.37 mmol) are added to the filtrate. The reaction mixture is stirred at 75° C. overnight and then cooled, diluted with water and extracted three times with ethyl acetate. The combined organic phases are washed with saturated sodium chloride solution and dried over sodium sulfate. The solvent is removed on a rotary evaporator, and the residue is dried under high vacuum. This gives 110 mg (98% of theory) of the title compound which is used without further purification for the next reaction step.

LC/MS (Method 6): Rₜ=2.75 min.; MS (ESIpos): m/z=302 [M+H]⁺.

### Example 29A

### N-(2-Amino-2-oxoethyl)-1-(2-fluorobenzyl)-1H-pyrazolo[3,4-b]pyridine-3-carboxamide

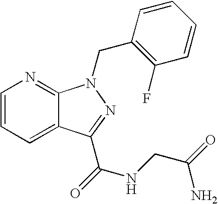

### Step a)

### 1-(2-Fluorobenzyl)-1H-pyrazolo[3,4-b]pyridine-3-carboxylic acid

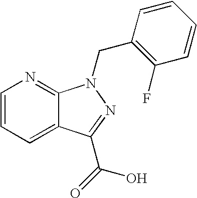

10.0 g of ethyl 1-(2-fluorobenzyl)-1H-pyrazolo[3,4-b]pyridine-3-carboxylate (33.41 mmol; prepared according to WO 03/095451, Example 2A) are dissolved in 500 ml of dioxane/water (1:1), and 50 ml of 1 N aqueous sodium hydroxide solution (50 mmol) are added. The reaction mixture is stirred at room temperature for 2 h and then acidified with 1 N hydrochloric acid, whereupon the product precipitates out. The product is filtered off and dried under high vacuum. This gives 8.71 g (96% of theory) of the title compound.

¹H-NMR (400 MHz, DMSO-d₆): δ=5.84 (s, 2H), 7.14-7.18 (m, 1H), 7.21-7.26 (m, 2H), 7.35-7.41 (m, 1H), 7.45 (dd, J=8.1, 4.6 Hz, 1H), 8.49 (dd, J=8.1, 1.5 Hz, 1H), 8.69 (dd, J=4.4, 1.2 Hz, 1H), 13.40 (br. s, 1H).

LC/MS (Method 2): Rₜ=1.69 min.; MS (ESIpos): m/z=272 [M+H]⁺.

### Step b)

### N-(2-Amino-2-oxoethyl)-1-(2-fluorobenzyl)-1H-pyrazolo[3,4-b]pyridine-3-carboxamide

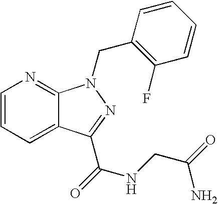

500 mg (1.84 mmol) of 1-(2-fluorobenzyl)-1H-pyrazolo[3,4-b]pyridine-3-carboxylic acid are suspended in 2 ml of dichloromethane. 1.67 g (12.9 mmol) of N-ethyldiisopropylamine and then 305 mg (2.77 mmol) of glycinamide hydrochloride, 374 mg (2.77 mmol) of HOBt and 570 mg (2.77 mmol) of DCC are added. The mixture is stirred at room temperature for 20 h and then diluted with dichloromethane and water, and the phases are separated. The organic phase is concentrated, and the residue is purified by preparative HPLC. This gives 409 mg (68% of theory) of the title compound.

¹H-NMR (400 MHz, DMSO-d₆): δ=3.86 (d, J=5.9 Hz, 2H), 5.84 (s, 2H), 7.09 (br. s, 1H), 7.12-7.27 (m, 3H), 7.33-7.45 (m, 3H), 8.39 (t, J=5.7 Hz, 1H), 8.57 (dd, J=8.1, 1.5 Hz, 1H), 8.68 (dd, J=4.4, 1.5 Hz, 1H).

LC/MS (Method 2): Rₜ=1.51 min.; MS (ESIpos): m/z=328 [M+H]⁺.

### Working Examples

### Example 1

### 3-[1-(2-Fluorobenzyl)-1H-pyrazolo[3,4-b]pyridin-3-yl]-1,2,4-oxadiazol-5(4H)-one

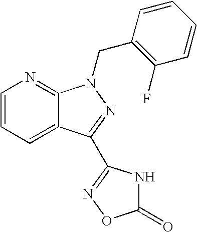

718 mg of 1-(2-fluorobenzyl)-N′-hydroxy-1H-pyrazolo[3,4-b]pyridine-3-carboximidamide (1.980 mmol, crude product from Example 1A) are dissolved in 10 ml of DMF, and 0.176 ml of pyridine (172 mg, 2.178 mmol) is added. At 0° C., 0.431 ml of 2-ethylhexyl chloroformate (402 mg, 1.980 mmol, 95% pure) is added dropwise, and the mixture is stirred at 0° C. for 40 min. 20 ml of water are added, and the reaction mixture is extracted three times with ethyl acetate. The combined organic phases are dried over sodium sulfate and concentrated on a rotary evaporator. The residue is taken up in 25 ml of xylene (isomer mixture) and heated under reflux for 16 h. The mixture is then cooled to room temperature, and the precipitate formed is filtered off. In this manner, 92 mg of product are isolated. The filtrate is concentrated on a rotary evaporator, 10 ml of xylene (isomer mixture) are added to the residue and the mixture is heated under reflux for another 16 h. The precipitate formed after cooling is filtered off, giving a further 201 mg of product. In this manner, a total of 293 mg (48% of theory) of the target compound are obtained.

¹H-NMR (400 MHz, CDCl₃): δ=5.88 (s, 2H), 7.04-7.18 (m, 3H), 7.26-7.36 (m, 2H), 8.50 (dd, J=8.1 and 1.5, 1H), 8.68 (dd, J=4.5 and 1.5, 1H), 12.67 (br. s, 1H).

LC/MS (Method 1): Rₜ=2.15 min.; MS (ESIpos): m/z=312 [M+H]⁺.

### Example 2

### 3-[1-(2-Fluorobenzyl)-1H-pyrazolo[3,4-b]pyridin-3-yl]-1,2,4-thiadiazol-5(4H)-one

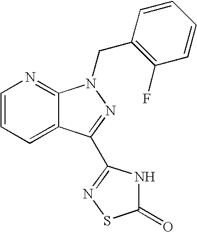

518 mg of 1-(2-fluorobenzyl)-N′-hydroxy-1H-pyrazolo[3,4-b]pyridine-3-carboximidamide (1.720 mmol, crude product from Example 1A) are dissolved in 10 ml of THF, 409 mg of N,N′-thiocarbonyldiimidazole (2.064 mmol, 90% pure) are added and the mixture is stirred at room temperature for 1 h. A further 204 mg of N,N′-thiocarbonyldiimidazole (1.030 mmol) are added, and the reaction mixture is stirred at room temperature for 45 min. 50 ml of water are added, and the mixture is extracted with a mixture of ethyl acetate, dichloromethane and THF. The combined organic phases are dried over sodium sulfate and concentrated on a rotary evaporator, and the residue is dried under high vacuum. Under an atmosphere of argon, the residue is then dissolved in 10 ml of dry THF, and 1.09 ml of boron trifluoride/diethyl etherate (1.221 g, 8.600 mmol) are added. After 16 h of stirring at room temperature, 20 ml of water are added and the precipitate formed is filtered off. The solid is triturated with dichloromethane, filtered off and dried under high vacuum. This gives 250 mg (44% of theory) of the target compound.

¹H-NMR (400 MHz, CDCl₃): δ=5.87 (s, 2H), 7.12-7.26 (m, 3H), 7.34-7.40 (m, 1H), 7.45-7.48 (m, 1H), 8.63 (dd, J=8.1 and 1.4, 1H), 8.72 (dd, J=4.5 and 1.4, 1H), 13.71 (br. s, 1H).

LC/MS (Method 3): Rₜ=2.38 min.; MS (ESIpos): m/z=328 [M+H]⁺.

### Example 3

### 5-[1-(2-Fluorobenzyl)-1H-pyrazolo[3,4-b]pyridin-3-yl]-1,3,4-oxadiazol-2(3H)-one

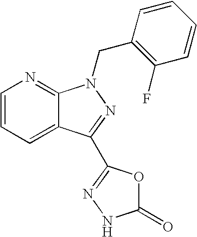

239 mg of 1-(2-fluorobenzyl)-1H-pyrazolo[3,4-b]pyridine-3-carbohydrazide (0.838 mmol, crude product from Example 2A) are dissolved in 5 ml of THF, 163 mg of N,N′-carbonyldiimidazole (1.003 mmol) are added and the mixture is heated under reflux for 1.5 h. After cooling of the mixture, 10 ml of water are added and the mixture is extracted three times with dichloromethane. The combined organic phases are washed twice with 1 N hydrochloric acid and with saturated aqueous sodium chloride solution and dried over sodium sulfate. The organic phase is concentrated on a rotary evaporator, and the residue is dried under high vacuum. The residue is then triturated with ethyl acetate, and the precipitate that remains is filtered off and dried under high vacuum. This gives 129 mg (49% of theory) of the target compound.

¹H-NMR (400 MHz, DMSO-d₆): δ=5.84 (s, 2H), 7.15-7.30 (m, 3H), 7.35-7.41 (m, 1H), 7.47 (dd, J=8.2 and 4.5, 1H), 8.44-8.46 (m, 1H), 8.74-8.76 (m, 1H), 12.81 (br. s, 1H).

LC/MS (Method 2): Rₜ=1.87 min.; MS (ESIpos): m/z=312 [M+H]⁺.

### Example 4

### 5-[1-(2-Fluorobenzyl)-1H-pyrazolo[3,4-b]pyridin-3-yl]-2-methyl-2,4-dihydro-3H-1,2,4-triazol-3-one

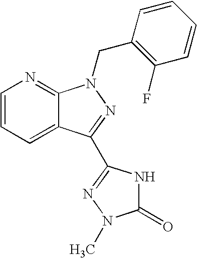

109 mg of 4-(2,4-dimethoxybenzyl)-5-[1-(2-fluorobenzyl)-1H-pyrazolo[3,4-b]pyridin-3-yl]-2-methyl-2,4-dihydro-3H-1,2,4-triazol-3-one (0.216 mmol, Example 6A) are dissolved in 3 ml of toluene and, with 5 mg of p-toluenesulfonic acid, heated under reflux for 16 h. After cooling, the solvent is removed on a rotary evaporator and the residue is purified by preparative HPLC (mobile phase: acetonitrile/water with 0.1% trifluoroacetic acid, gradient 20:80→95:5). This gives 28 mg of the title compound (40% of theory).

¹H-NMR (400 MHz, DMSO-d₆): δ=3.41 (s, 3H), 5.81 (s, 2H), 7.11-7.25 (m, 3H), 7.33-7.44 (m, 2H), 8.52 (dd, J=8.1 and 1.4, 1H), 8.70 (dd, J=4.4 and 1.4, 1H), 12.39 (br. s, 1H).

LC/MS (Method 3): Rₜ=2.06 min.; MS (ESIpos): m/z=325 [M+H]⁺.

### Example 5

### 2-Ethyl-5-[1-(2-fluorobenzyl)-1H-pyrazolo[3,4-b]pyridin-3-yl]-2,4-dihydro-3H-1,2,4-triazol-3-one

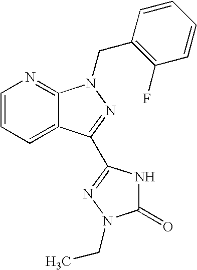

76 mg of 4-(2,4-dimethoxybenzyl)-2-ethyl-5-[1-(2-fluorobenzyl)-1H-pyrazolo[3,4-b]pyridin-3-yl]-2,4-dihydro-3H-1,2,4-triazol-3-one (0.129 mmol, Example 7A, 83% pure) are dissolved in 3 ml of toluene and, with 5 mg of p-toluenesulfonic acid, heated under reflux for 16 h. After cooling, the solvent is removed on a rotary evaporator and the residue is purified by preparative HPLC (mobile phase: acetonitrile/water with 0.1% trifluoroacetic acid, gradient 20:80→95:5). This gives 31 mg of the title compound (71% of theory).

¹H-NMR (400 MHz, DMSO-d₆): δ=1.30 (t, J=7.2, 3H), 3.80 (q, J=7.2, 2H), 5.82 (s, 2H), 7.11-7.26 (m, 3H), 7.32-7.44 (m, 2H), 8.54 (dd, J=8.1 and 1.5, 1H), 8.70 (dd, J=4.4 and 1.5, 1H), 12.37 (br. s, 1H).

LC/MS (Method 1): Rₜ=2.05 min.; MS (ESIpos): m/z=339 [M+H]⁺.

### Example 6

### 5-[1-(2-Fluorobenzyl)-1H-pyrazolo[3,4-b]pyridin-3-yl]-4-isopropyl-2,4-dihydro-3H-1,2,4-triazol-3-one

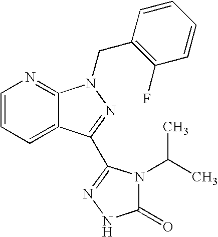

50 mg of 2-{[1-(2-fluorobenzyl)-1H-pyrazolo[3,4-b]pyridin-3-yl]carbonyl}-N-isopropylhydrazine-carboxamide (0.135 mmol, Example 8A) are suspended in 1 ml of 2% strength aqueous sodium hydroxide solution, and the mixture is heated under reflux for 72 h. After cooling, the reaction mixture is acidified with 1 N hydrochloric acid and extracted with ethyl acetate. The combined organic phases are dried over sodium sulfate, and the solvent is removed on a rotary evaporator. The residue is purified by preparative HPLC (mobile phase: acetonitrile/water with 0.1% trifluoroacetic acid, gradient 20:80→95:5). This gives 7 mg of the title compound (15% of theory).

¹H-NMR (400 MHz, DMSO-d₆): δ=1.41 (d, J=6.8, 6H), 5.10 (sept, J=6.8, 1H), 5.81 (s, 2H), 7.15-7.26 (m, 2H), 7.31-7.42 (m, 3H), 8.48-8.50 (m, 1H), 8.70-8.72 (m, 1H), 12.10 (br. s, 1H).

LC/MS (Method 2): Rₜ=2.02 min.; MS (ESIpos): m/z=353 [M+H]⁺.

### Example 7

### 3-[1-(2-Fluorobenzyl)-1H-pyrazolo[3,4-b]pyridin-3-yl]-1,2,4-oxadiazole-5(4H)-thione

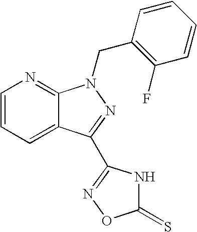

650 mg of 1-(2-fluorobenzyl)-N′-hydroxy-1H-pyrazolo[3,4-b]pyridine-3-carboximidamide (1.980 mmol, crude product from Example 1A) are dissolved in 20 ml of acetonitrile, and 842 mg of N,N′-thiocarbonyldiimidazole (4.360 mmol) and 950 μl of 1,5-diazabicyclo[4.3.0]non-5-ene (985 mg, 7.928 mmol) are added. The reaction mixture is stirred at room temperature for 24 h. For work-up, the reaction mixture is concentrated on a rotary evaporator and the residue is taken up in ethyl acetate. The solution is washed three times with 5% strength citric acid and with saturated sodium chloride solution. The organic phase is dried over sodium sulfate, the solvent is removed on a rotary evaporator and the residue is triturated with dichloromethane. The precipitate is filtered off and dried under high vacuum. This gives 417 mg (64% of theory) of the target compound.

¹H-NMR (400 MHz, DMSO-d₆): δ=5.81 (s, 2H), 7.07-7.22 (m, 3H), 7.28-7.33 (m, 1H), 7.41-7.44 (m, 1H), 8.39-8.41 (m, 1H), 8.69-8.70 (m, 1H).

LC/MS (Method 2): Rₜ=2.01 min.; MS (ESIpos): m/z=328 [M+H]⁺.

### Example 8

### 3-[1-(2,3-Difluorobenzyl)-1H-pyrazolo[3,4-b]pyridin-3-yl]-1,2,4-oxadiazol-5(4H)-one

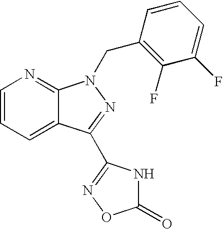

65 mg of 1-(2,3-difluorobenzyl)-N′-hydroxy-1H-pyrazolo[3,4-b]pyridine-3-carboximidamide (0.21 mmol, crude product from Example 9A) are dissolved in 1.3 ml of DMF, and 19 μl of pyridine (0.24 mmol) are added. At 0° C., 28 μl of isobutyl chloroformate (0.21 mmol) are added dropwise. The reaction mixture is stirred initially at 0° C. for 40 min and then at 200° C. in a microwave oven for 2 h. After cooling, the reaction mixture is purified directly by preparative HPLC (Method 5). This gives 37 mg (52% of theory) of the title compound.

LC/MS (Method 1): Rₜ=2.26 min.; MS (ESIpos): m/z=330 [M+H]⁺

¹H-NMR (400 MHz, CDCl₃): δ=5.92 (s, 2H), 7.05 (dd, 1H), 7.17 (dd, 1H), 7.41 (dd, 1H), 7.50 (dd, 1H), 8.47 (dd, 1H), 8.77 (dd, 1H), 13.35 (br. s, 1H).

### 3-[1′-(2,5-Difluorobenzyl)-1H-pyrazolo[3,4-b]pyridin-3-yl]-1,2,4-oxadiazol-5(4H)-one

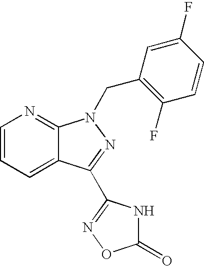

84 mg of 1-(2,5-difluorobenzyl)-N′-hydroxy-1H-pyrazolo[3,4-b]pyridine-3-carboximidamide (0.28 mmol, crude product from Example 10A) are dissolved in 1.7 ml of DMF, and 25 μl of pyridine (0.31 mmol) are added. At 0° C., 36 μl of isobutyl chloroformate (37.8 mg, 0.28 mmol) are added dropwise. The reaction mixture is stirred at 0° C. for 40 min, 20 ml of water are then added and the mixture is extracted three times with ethyl acetate. The combined organic phases are washed once with saturated sodium chloride solution, dried over sodium sulfate and concentrated on a rotary evaporator. The residue is dissolved in 4 ml of xylene and 200 μl of 1-n-butyl-3-methylimidazolium hexafluorophosphate and stirred at 200° C. in a microwave oven for 2 h. After cooling, the reaction mixture is concentrated on a rotary evaporator. The residue is dissolved in DMSO and purified by preparative HPLC (Method 5). This gives 60 mg (66% of theory) of the title compound.

LC/MS (Method 2): Rₜ=2.03 min.; MS (ESIpos): m/z=330 [M+H]⁺

¹H-NMR (400 MHz, CDCl₃): δ=5.86 (s, 2H), 7.11-7.17 (m, 1H), 7.20-7.27 (m, 1H), 7.28-7.35 (m, 1H), 7.50 (dd, 1H), 8.46 (dd, 1H), 8.77 (dd, 1H), 13.32 (br. s, 1H).

### Example 10

### 3-[1-(2,6-Difluorobenzyl)-1H-pyrazolo[3,4-b]pyridin-3-yl]-1,2,4-oxadiazol-5(4H)-one

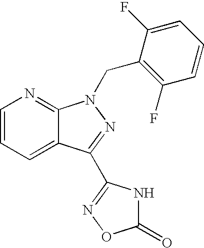

100 mg of 1-(2,6-difluorobenzyl)-N′-hydroxy-1H-pyrazolo[3,4-b]pyridine-3-carboximidamide (0.33 mmol, crude product from Example 11A) are dissolved in 2 ml of DMF, and 29 μl of pyridine (28.7 mg, 0.36 mmol) are added. At 0° C., 43 μl of isobutyl chloroformate (45 mg, 0.33 mmol) are added dropwise. The reaction mixture is stirred at 0° C. for 40 min, 20 ml of water are then added and the mixture is extracted three times with ethyl acetate. The combined organic phases are washed once with saturated sodium chloride solution, dried over sodium sulfate and concentrated on a rotary evaporator. The residue is dissolved in 10 ml of xylene and stirred under reflux for 3 days. After cooling, the reaction mixture is concentrated on a rotary evaporator. The residue is dissolved in DMSO and purified by preparative HPLC (Method 5). This gives 47 mg (43% of theory) of the title compound.

¹H-NMR (400 MHz, CDCl₃): δ=5.87 (s, 2H), 7.11-7.18 (m, 2H), 7.44-7.52 (m, 2H), 8.44 (dd, 1H), 8.77 (dd, 1H), 13.30 (br. s, 1H).

LC/MS (Method 2): Rₜ=1.99 min.; MS (ESIpos): m/z=330 [M+H]⁺.

### Example 11

### 3-{1-[(5-Chloro-2-thienyl)methyl]-1H-pyrazolo[3,4-b]pyridin-3-yl}-1,2,4-oxadiazol-5(4H)-one

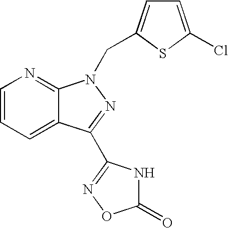

Analogously to Example 9, 1-[(5-chloro-2-thienyl)methyl]-N′-hydroxy-1H-pyrazolo[3,4-b]pyridine-3-carboximidamide (60 mg, 0.20 mmol; crude product from Example 12A) gives 42 mg (65% of theory) of the title compound.

¹H-NMR (400 MHz, CDCl₃): δ=5.94 (s, 2H), 7.00 (d, 1H), 7.08 (d, 1H), 7.50 (dd, 1H), 8.46 (dd, 1H), 8.79 (dd, 1H), 13.38 (br. s, 1H).

LC/MS (Method 2): Rₜ=2.12 min.; MS (ESIpos): m/z=334 [M+H]⁺.

### Example 12

### 5-[1-(2-Fluorobenzyl)-1H-pyrazolo[3,4-b]pyridin-3-yl]-2,4-dihydro-3H-1,2,4-triazol-3-one

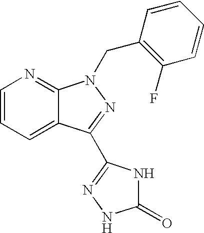

53 mg of 4-(2,4-dimethoxybenzyl)-5-[1-(2-fluorobenzyl)-1H-pyrazolo[3,4-b]pyridin-3-yl]-2,4-dihydro-3H-1,2,4-triazol-3-one (0.114 mmol; crude product from Example 5A, 97% pure) are dissolved in 2 ml of toluene and, with 5 mg of p-toluenesulfonic acid, heated under reflux for 16 h. After cooling, the precipitate formed is filtered off and taken up in 1 N aqueous sodium hydroxide solution. Undissolved components are filtered off, and the filtrate is extracted with ethyl acetate. The extract is acidified with 1 N hydrochloric acid, the precipitate formed is filtered off and the residue is dried under high vacuum. This gives 13 mg (36% of theory) of the title compound.

¹H-NMR (400 MHz, DMSO-d₆): δ=5.81 (s, 2H), 7.11-7.17 (m, 2H), 7.21-7.26 (m, 1H), 7.33-7.42 (m, 2H), 8.49-8.51 (m, 1H), 8.68-8.69 (m, 1H), 11.85 (s, 1H), 12.18 (s, 1H).

LC/MS (Method 1): Rₜ=1.74 min.; MS (ESIpos): m/z=311 [M+H]⁺.

### Example 13

### 5-[1-(2-Fluorobenzyl)-1H-pyrazolo[3,4-b]pyridin-3-yl]-2-propyl-2,4-dihydro-3H-1,2,4-triazol-3-one

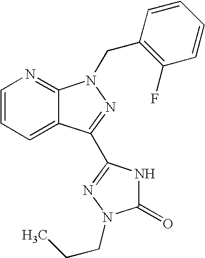

4-(2,4-Dimethoxybenzyl)-5-[1-(2-fluorobenzyl)-1H-pyrazolo[3,4-b]pyridin-3-yl]-2-propyl-2,4-dihydro-3H-1,2,4-triazol-3-one (Example 13A; 205 mg, 0.41 mmol) is, in a mixture of concentrated sulfuric acid (1.5 ml) and glacial acetic acid (4.0 ml), warmed at 50° C. for 4 h. The mixture is then stirred into ice-water, made alkaline with saturated sodium carbonate solution and extracted with ethyl acetate. The organic phase is concentrated, and the residue is purified by preparative HPLC. This gives 112 mg (78% of theory) of the title compound.

¹H-NMR (400 MHz, DMSO-d₆): δ=0.90 (t, J=7.5 Hz, 3H), 1.75 (tq, J=7.5, 6.8 Hz, 2H), 3.72 (t, J=6.8 Hz, 2H), 5.82 (s, 2H), 7.10-7.17 (m, 2H), 7.20-7.26 (m, 1H), 7.32-7.39 (m, 1H), 7.42 (dd, J=8.1, 4.4 Hz, 1H), 8.52 (dd, J=8.1, 1.5 Hz, 1H), 8.70 (dd, J=4.4, 1.5 Hz, 1H), 12.37 (br. s, 1H).

LC/MS (Method 2): Rₜ=2.08 min.; MS (ESIpos): m/z=353 [M+H]⁺.

### Example 14

### 5-[1-(2-Fluorobenzyl)-1H-pyrazolo[3,4-b]pyridin-3-yl]-2-isobutyl-2,4-dihydro-3H-1,2,4-triazol-3-one

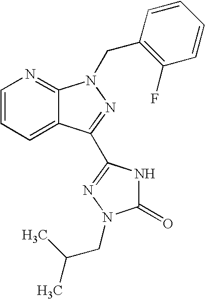

The title compound is synthesized analogously to Example 13 from 4-(2,4-dimethoxybenzyl)-5-[1-(2-fluorobenzyl)-1H-pyrazolo[3,4-b]pyridin-3-yl]-2-isobutyl-2,4-dihydro-3H-1,2,4-triazol-3-one (Example 14A; 209 mg, 0.405 mmol). The product is purified by preparative HPLC, giving 100 mg (67% of theory) of the title compound as a white solid.

¹H-NMR (400 MHz, DMSO-d₆): δ=0.92 (d, J=6.6 Hz, 6H), 2.07-2.20 (m, 1H), 3.58 (d, J=7.1 Hz, 2H), 5.82 (s, 2H), 7.10-7.18 (m, 2H), 7.20-7.26 (m, 1H), 7.32-7.39 (m, 1H), 7.42 (dd, J=8.1, 4.4 Hz, 1H), 8.51 (dd, J=8.1, 1.5 Hz, 1H), 8.70 (dd, J=4.4, 1.5 Hz, 1H), 12.38 (br. s, 1H).

LC/MS (Method 1): Rₜ=2.37 min.; MS (ESIpos): m/z=367 [M+H]⁺.

### Example 15

### 5-[1-(2-Fluorobenzyl)-1H-pyrazolo[3,4-b]pyridin-3-yl]-2-isopropyl-2,4-dihydro-3H-1,2,4-triazol-3-one

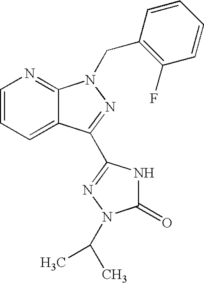

The title compound is synthesized analogously to Example 13 from 4-(2,4-dimethoxybenzyl)-5-[1-(2-fluorobenzyl)-1H-pyrazolo[3,4-b]pyridin-3-yl]-2-isopropyl-2,4-dihydro-3H-1,2,4-triazol-3-one (Example 15A; 205 mg, 0.408 mmol). The product is purified by preparative HPLC, giving 108 mg (75% of theory) of the title compound as a white solid.

¹H-NMR (400 MHz, DMSO-d₆): δ=1.36 (d, J=6.6 Hz, 6H), 4.39 (sept, J=6.6 Hz, 1H), 5.82 (s, 2H), 7.09-7.15 (m, 2H), 7.20-7.26 (m, 1H), 7.32-7.39 (m, 1H), 7.43 (dd, J=8.1, 4.4 Hz, 1H), 8.55 (dd, J=8.1, 1.5 Hz, 1H), 8.70 (dd, J=4.4, 1.5 Hz, 1H), 12.34 (br. s, 1H).

LC/MS (Method 2): Rₜ=2.10 min.; MS (ESIpos): m/z=353 [M+H]⁺.

### Example 16

### 2-(2-Fluoroethyl)-5-[1-(2-fluorobenzyl)-1H-pyrazolo[3,4-b]pyridin-3-yl]-2,4-dihydro-3H-1,2,4-triazol-3-one

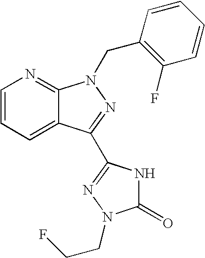

The title compound is synthesized analogously to Example 13 from 4-(2,4-dimethoxybenzyl)-5-[1-(2-fluorobenzyl)-1H-pyrazolo[3,4-b]pyridin-3-yl]-2-(2-fluoroethyl)-2,4-dihydro-3H-1,2,4-triazol-3-one (Example 16A; 215 mg, 0.424 mmol). The product is purified by preparative HPLC, giving 62 mg (41% of theory) of the title compound as a white solid.

¹H-NMR (400 MHz, DMSO-d₆): δ=4.08 (dt, J=26.7, 4.7 Hz, 2H), 4.76 (dt, J=47.2, 4.7 Hz, 2H), 5.83 (s, 2H), 7.10-7.17 (m, 2H), 7.20-7.27 (m, 1H), 7.32-7.39 (m, 1H), 7.43 (dd, J=8.1, 4.4 Hz, 1H), 8.54 (dd, J=8.1, 1.5 Hz, 1H), 8.71 (dd, J=4.4, 1.5 Hz, 1H), 12.49 (br. s, 1H).

LC/MS (Method 2): Rₜ=1.85 min.; MS (ESIpos): m/z=357 [M+H]⁺.

### Example 17

### 2-(2,2-Difluoroethyl)-5-[1-(2-fluorobenzyl)-1H-pyrazolo[3,4-b]pyridin-3-yl]-2,4-dihydro-3H-1,2,4-triazol-3-one

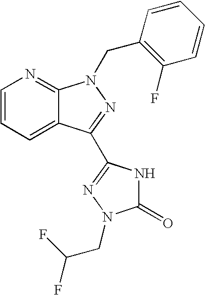

The title compound is synthesized analogously to Example 13 from 4-(2,4-dimethoxybenzyl)-5-[1-(2-fluorobenzyl)-1H-pyrazolo[3,4-b]pyridin-3-yl]-2-(2,2-difluoroethyl)-2,4-dihydro-3H-1,2,4-triazol-3-one (Example 17A; 239 mg, 0.456 mmol). The product is purified by preparative HPLC, giving 107 mg (63% of theory) of the title compound as a white solid.

¹H-NMR (400 MHz, DMSO-d₆): δ=4.23 (dt, J=15.2, 3.5 Hz, 2H), 5.83 (s, 2H), 6.39 (tt, J=58.4, 3.5 Hz, 1H), 7.10-7.17 (m, 2H), 7.20-7.26 (m, 1H), 7.33-7.39 (m, 1H), 7.44 (dd, J=8.1, 4.4 Hz, 1H), 8.53 (dd, J=8.1, 1.5 Hz, 1H), 8.71 (dd, J=4.4, 1.5 Hz, 1H), 12.63 (br. s, 1H).

LC/MS (Method 2): Rₜ=1.98 min.; MS (ESIpos): m/z=375 [M+H]⁺.

### Example 18

### 5-[1-(2-Fluorobenzyl)-1H-pyrazolo[3,4-b]pyridin-3-yl]-2-(2,2,2-trifluoroethyl)-2,4-dihydro-3H-1,2,4-triazol-3-one

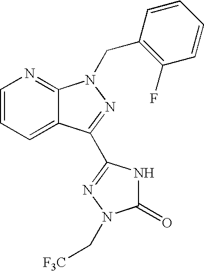

4-(2,4-Dimethoxybenzyl)-5-[1-(2-fluorobenzyl)-1H-pyrazolo[3,4-b]pyridin-3-yl]-2-(2,2,2-trifluoroethyl)-2,4-dihydro-3H-1,2,4-triazol-3-one (Example 18A; 179 mg, 0.33 mmol) is, in a mixture of concentrated sulfuric acid (1.0 ml) and glacial acetic acid (3.0 ml), warmed at 40° C. for 5 h. The mixture is then stirred into ice-water, made alkaline with saturated sodium carbonate solution and extracted with ethyl acetate. The organic phase is concentrated, and the residue is purified by preparative HPLC. This gives 77 mg (59% of theory) of the title compound as a white solid.

¹H-NMR (400 MHz, DMSO-d₆): δ=4.70 (q, J=9.1 Hz, 2H), 5.84 (s, 2H), 7.10-7.19 (m, 2H), 7.23 (dd, J=10.3, 8.3 Hz, 1H), 7.32-7.40 (m, 1H), 7.46 (dd, J=8.1, 4.4 Hz, 1H), 8.48 (dd, J=8.1, 1.5 Hz, 1H), 8.72 (dd, J=4.4, 1.5 Hz, 1H), 12.75 (s, 1H).

LC/MS (Method 1): Rₜ=2.31 min.; MS (ESIpos): m/z=393 [M+H]⁺.

### Example 19

### 2-{3-[1-(2-Fluorobenzyl)-1H-pyrazolo[3,4-b]pyridin-3-yl]-5-oxo-4,5-dihydro-1H-1,2,4-triazol-1-yl}ethyl acetate

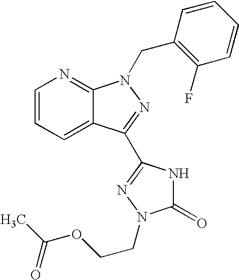

4-(2,4-Dimethoxybenzyl)-5-[1-(2-fluorobenzyl)-1H-pyrazolo[3,4-b]pyridin-3-yl]-2-(3-hydroxyethyl)-2,4-dihydro-3H-1,2,4-triazol-3-one (Example 19A; 210 mg, 0.42 mmol) is, in a mixture of concentrated sulfuric acid (0.5 ml) and glacial acetic acid (4.0 ml), warmed at 40° C. for 5 h. After addition of further sulfuric acid (0.5 ml), the mixture is stirred at 40° C. for another 5 h. The mixture is then stirred into ice-water, made alkaline with saturated sodium carbonate solution and extracted with ethyl acetate. The organic phase is concentrated, and the residue is purified by preparative HPLC. This gives 83 mg (50% of theory) of the title compound as a white solid.

¹H-NMR (400 MHz, DMSO-d₆): δ=1.97 (s, 3H), 4.00 (t, J=5.2 Hz, 2H), 4.34 (t, J=5.2 Hz, 2H), 5.81 (s, 2H), 7.10-7.18 (m, 2H), 7.23 (dd, J=10.3, 8.3 Hz, 1H), 7.32-7.39 (m, 1H), 7.42 (dd, J=8.1, 4.4 Hz, 1H), 8.53 (dd, J=8.1, 1.4 Hz, 1H), 8.69 (dd, J=4.4, 1.4 Hz, 1H), 12.42 (br. s, 1H).

LC/MS (Method 4): Rₜ=2.20 min.; MS (ESIpos): m/z=397 [M+H]⁺.

### Example 20

### 3-{3-[1-(2-Fluorobenzyl)-1H-pyrazolo[3,4-b]pyridin-3-yl]-5-oxo-4,5-dihydro-1H-1,2,4-triazol-1-yl}propyl acetate

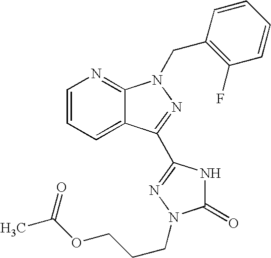

4-(2,4-Dimethoxybenzyl)-5-[1-(2-fluorobenzyl)-1H-pyrazolo[3,4-b]pyridin-3-yl]-2-(3-hydroxypropyl)-2,4-dihydro-3H-1,2,4-triazol-3-one (Example 20A; 130 mg, 0.25 mmol) is, in a mixture of concentrated sulfuric acid (0.5 ml) and glacial acetic acid (2.0 ml), warmed at 40° C. for 5 h. The mixture is then stirred into ice-water, made alkaline with saturated sodium carbonate solution and extracted with ethyl acetate. The organic phase is concentrated, and the residue is purified by preparative HPLC. This gives 75 mg (73% of theory) of the title compound as a solid.

¹H-NMR (400 MHz, DMSO-d₆): δ=1.99 (s, 3H), 2.03 (tt, J=6.6, 6.4 Hz, 2H), 3.82 (t, J=6.6 Hz, 2H), 4.07 (t, J=6.4 Hz, 2H), 5.80 (s, 2H), 7.10-7.15 (m, 2H), 7.23 (dd, J=10.3, 8.3 Hz, 1H), 7.32-7.38 (m, 1H), 7.39 (dd, J=8.1, 4.4 Hz, 1H), 8.55 (dd, J=8.1, 1.5 Hz, 1H), 8.67 (dd, J=4.4, 1.5 Hz, 1H), 12.40 (br. s, 1H).

LC/MS (Method 2): Rₜ=1.92 min.; MS (ESIpos): m/z=411 [M+H]⁺.

### Example 21

### 5-[1-(2-Fluorobenzyl)-1H-pyrazolo[3,4-b]pyridin-3-yl]-2-(3-hydroxypropyl)-2,4-dihydro-3H-1,2,4-triazol-3-one

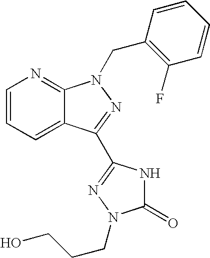

3-{3-[1-(2-Fluorobenzyl)-1H-pyrazolo[3,4-b]pyridin-3-yl]-5-oxo-4,5-dihydro-1H-1,2,4-triazol-1-yl}propyl acetate (Example 20; 60 mg, 0.15 mmol) is dissolved in methanol (9.0 ml) with addition of a 5.4 M sodium methoxide solution (3.0 ml) and stirred at RT for 20 h. A pH of 7 is then adjusted using concentrated hydrochloric acid. Precipitated salt is dissolved by addition of water, and the mixture is purified by preparative HPLC. This gives 40 mg (74% of theory) of the title compound as white crystals.

¹H-NMR (400 MHz, DMSO-d₆): δ=1.88 (tt, J=7.1, 6.3 Hz, 2H), 3.49 (dt, J=6.3, 4.9 Hz, 2H), 3.82 (t, J=7.1 Hz, 2H), 4.57 (t, J=4.9 Hz, 1H), 5.82 (s, 2H), 7.10-7.17 (m, 2H), 7.23 (dd, J=10.3, 8.3 Hz, 1H), 7.23-7.39 (m, 1H), 7.42 (dd, J=8.1, 4.5 Hz, 1H), 8.53 (dd, J=8.1, 1.5 Hz, 1H), 8.70 (dd, J=4.5, 1.5 Hz, 1H), 12.38 (br. s, 1H).

LC/MS (Method 1): Rₜ=1.78 min.; MS (ESIpos): m/z=369 [M+H]⁺.

### Example 22

### 5-[1-(2-Fluorobenzyl)-1H-pyrazolo[3,4-b]pyridin-3-yl]-2-(2-hydroxyethyl)-2,4-dihydro-3H-1,2,4-triazol-3-one

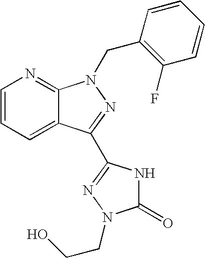

2-{3-[1-(2-Fluorobenzyl)-1H-pyrazolo[3,4-b]pyridin-3-yl]-5-oxo-4,5-dihydro-1H-1,2,4-triazol-1-yl}ethyl acetate (Example 19; 70 mg, 0.18 mmol) is dissolved in methanol (9.0 ml) with addition of a 5.4 M sodium methoxide solution (3.0 ml) and stirred at RT for 20 h. A pH of 7 is then adjusted using concentrated hydrochloric acid. Precipitated salt is dissolved by addition of water, and the mixture is purified by preparative HPLC. This gives 56 mg (89% of theory) of the title compound as light-yellow crystals.

¹H-NMR (400 MHz, DMSO-d₆): δ=3.62-3.69 (m, 2H), 3.80 (t, J=5.5 Hz, 2H), 5.74 (s, 2H), 6.17 (br. s, 1H), 7.07-7.14 (m, 2H), 7.22 (dd, J=10.3, 8.3 Hz, 1H), 7.28 (dd, J=7.8, 4.4 Hz, 1H), 7.30-7.37 (m, 1H), 8.57 (dd, J=4.4, 1.5 Hz, 1H), 8.61 (dd, J=7.8, 1.5 Hz, 1H), 12.35 (br. s, 1H).

LC/MS (Method 2): Rₜ=1.59 min.; MS (ESIpos): m/z=355 [M+H]⁺.

### Example 23

### 5-[1-(2-Fluorobenzyl)-1H-pyrazolo[3,4-b]pyridin-3-yl]-2-(3-morpholin-4-ylpropyl)-2,4-dihydro-3H-1,2,4-triazol-3-one

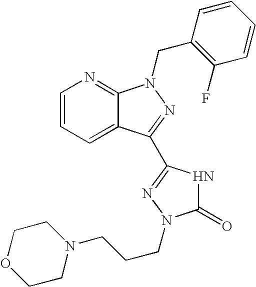

4-(2,4-Dimethoxybenzyl)-5-[1-(2-fluorobenzyl)-1H-pyrazolo[3,4-b]pyridin-3-yl]-2-(3-morpholin-4-ylpropyl)-2,4-dihydro-3H-1,2,4-triazol-3-one (Example 21A; 220 mg, 0.37 mmol) is dissolved in glacial acetic acid (3.0 ml), and concentrated sulfuric acid (0.8 ml) is added. The mixture is stirred at RT for 20 h and then at 40° C. for 8 h. The mixture is then added to ice-water, made alkaline with saturated sodium carbonate solution and extracted with ethyl acetate. The organic phase is dried over sodium sulfate and concentrated under reduced pressure. The residue is purified by preparative HPLC. This gives 39 mg (22% of theory) of the title compound as a white solid.

¹H-NMR (400 MHz, DMSO-d₆): δ=1.88 (quint, J=6.9 Hz, 2H), 2.31-2.37 (m, 6H), 3.53 (t, J=4.5 Hz, 4H), 3.80 (t, J=6.9 Hz, 2H), 5.82 (s, 2H), 7.10-7.17 (m, 2H), 7.23 (dd, J=10.3, 8.3 Hz, 1H), 7.33-7.39 (m, 1H), 7.42 (dd, J=8.1, 4.7 Hz, 1H), 8.52 (dd, J=8.1, 1.5 Hz, 1H), 8.70 (dd, J=4.7, 1.5 Hz, 1H), 12.35 (br. s, 1H).

LC/MS (Method 1): Rₜ=1.45 min.; MS (ESIpos): m/z=438 [M+H]⁺.

### Example 24

### 3-{3-[1-(2-Fluorobenzyl)-1H-pyrazolo[3,4-b]pyridin-3-yl]-5-oxo-4,5-dihydro-1H-1,2,4-triazol-1-yl}propanamide

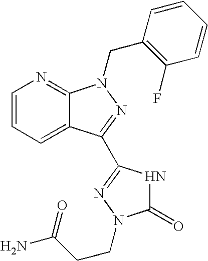

3-{4-(2,4-Dimethoxybenzyl)-3-[1-(2-fluorobenzyl)-1H-pyrazolo[3,4-b]pyridin-3-yl]-5-oxo-4,5-dihydro-1H-1,2,4-triazol-1-yl}propanenitrile (Example 22A; 155 mg, 0.30 mmol) is dissolved in glacial acetic acid (3.0 ml), and concentrated sulfuric acid (1.0 ml) is added. The solution is heated at 40° C. for 5 h, then diluted with water and, with cooling, adjusted to pH 6.5 using concentrated aqueous sodium hydroxide solution. The product is purified by preparative HPLC, giving 35 mg (30% of theory) of the title compound as a yellow crystalline product.

¹H-NMR (400 MHz, DMSO-d₆): δ=3.32 (s, 2H), 3.93 (t, J=7.2 Hz, 2H), 5.80 (s, 2H), 6.89 (br. s, 1H), 7.10-7.15 (m, 2H), 7.20-7.26 (m, 1H), 7.32-7.37 (m, 1H), 7.40 (dd, J=7.8, 4.4 Hz, 1H), 7.52 (br. s, 1H), 8.56 (dd, J=8.1, 1.2 Hz, 1H), 8.68 (dd, J=4.4, 1.2 Hz, 1H), 12.36 (br. s, 1H).

LC/MS (Method 2): Rₜ=1.49 min.; MS (ESIpos): m/z=382 [M+H]⁺.

### Example 25

### 3-[1-(3-Fluorobenzyl)-1H-pyrazolo[3,4-b]pyridin-3-yl]-1,2,4-oxadiazol-5(4H)-one

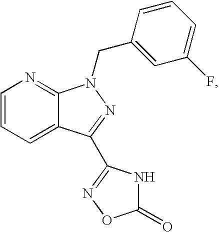

64 mg of 1-(3-fluorobenzyl)-N′-hydroxy-1H-pyrazolo[3,4-b]pyridin-3-carboximidamide (0.22 mmol, crude product from Example 23A) are dissolved in 1.4 ml of DMF, and 20 μl of pyridine (0.25 mmol) are added. At 0° C., 29 μl of isobutyl chloroformate (0.22 mmol) are added dropwise. The reaction mixture is stirred initially at 0° C. for 40 min and then at 200° C. in a microwave oven for 2 h. After cooling, the reaction mixture is purified by preparative HPLC (Method 5). This gives 41 mg (59% of theory) of the title compound.

LC/MS (Method 1): Rₜ=2.23 min.; MS (ESIpos): m/z=312 [M+H]⁺

¹H-NMR (400 MHz, DMSO-d₆): δ=5.84 (s, 2H), 7.08-7.18 (m, 3H), 7.38 (q, 1H), 7.50 (dd, 1H), 8.48 (dd, 1H), 8.77 (dd, 1H), 13.36 (br. s, 1H).

### Example 26

### 3-[1-(2,4-Difluorobenzyl)-1H-pyrazolo[3,4-b]pyridin-3-yl]-1,2,4-oxadiazol-5(4H)-one

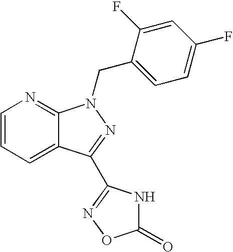

Using the same process as described for Example 25, 76 mg of 1-(2,4-difluorobenzyl)-N′-hydroxy-1H-pyrazolo[3,4-b]pyridine-3-carboximidamide (0.25 mmol, crude product from Example 24A) give 36 mg (43% of theory) of the title compound.

LC/MS (Method 1): Rₜ=2.27 min.; MS (ESIpos): m/z=330 [M+H]⁺

¹H-NMR (400 MHz, DMSO-d₆): δ=5.84 (s, 2H), 7.05 (dt, 1H), 7.25-7.39 (m, 2H) 7.50 (dd, 1H), 8.46 (dd, 1H), 8.77 (dd, 1H), 13.33 (br. s, 1H).

### Example 27

### 3-[1-(5-Chloro-2-fluorobenzyl)-1H-pyrazolo[3,4-b]pyridin-3-yl]-1,2,4-oxadiazol-5(4H)-one

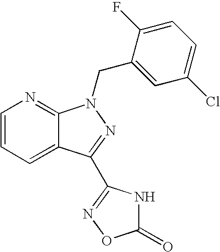

64 mg of 1-(5-chloro-2-fluorobenzyl)-N′-hydroxy-1H-pyrazolo[3,4-b]pyridine-3-carboximidamide (0.22 mmol, crude product from Example 25A) are dissolved in 1.4 ml of DMF, and 20 μl of pyridine (0.25 mmol) are added. At 0° C., 29 μl of isobutyl chloroformate (0.22 mmol) are added dropwise. The reaction mixture is stirred at 0° C. for 40 min, water is then added and the mixture is extracted three times with ethyl acetate. The combined organic phases are washed once with saturated sodium chloride solution, dried over sodium sulfate and concentrated on a rotary evaporator. The residue is dissolved in 3 ml of xylene and 200 μl of 1-n-butyl-3-methylimidazolium hexafluorophosphate and stirred at 200° C. in a microwave oven for 2 h. The reaction mixture is then concentrated on a rotary evaporator. The residue obtained is dissolved in DMSO and purified by preparative HPLC (Method 5). This gives 44 mg (57% of theory) of the title compound.

LC/MS (Method 1): Rₜ=2.33 min.; MS (ESIpos): m/z=346 [M+H]⁺

¹H-NMR (400 MHz, DMSO-d₆): δ=5.88 (s, 2H), 7.30 (t, 1H), 7.41 (dd, 1H), 7.44-7.52 (m, 2H), 8.46 (dd, 1H), 8.78 (dd, 1H), 13.33 (br. s, 1H).

### Example 28

### 3-[1-(2-Fluoro-3-methylbenzyl)-1H-pyrazolo[3,4-b]pyridin-3-yl]-1,2,4-oxadiazol-5(4H)-one

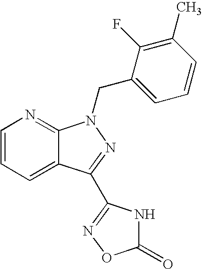

Using the same process as described for Example 27, 57 mg of 1-(2-fluoro-3-methylbenzyl)-N′-hydroxy-1H-pyrazolo[3,4-b]pyridine-3-carboximidamide (0.19 mmol, crude product from Example 26A) give 22 mg (36% of theory) of the title compound.

LC/MS (Method 1): Rₜ=2.31 min.; MS (ESIpos): m/z=326 [M+H]⁺

¹H-NMR (400 MHz, DMSO-d₆): δ=2.22 (s, 3H), 5.85 (s, 2H), 6.97-7.05 (m, 2H), 7.22 (m, 1H), 7.49 (dd, 1H), 8.46 (dd, 1H), 8.78 (dd, 1H), 13.32 (br. s, 1H).

### Example 29

### 3-[1-(Cycloheptylmethyl)-1H-pyrazolo[3,4-b]pyridin-3-yl]-1,2,4-oxadiazol-5(4H)-one

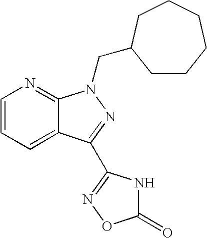

316 mg of 1-(cycloheptylmethyl)-N′-hydroxy-1H-pyrazolo[3,4-b]pyridine-3-carboximidamide (1.101 mmol, crude product from Example 27A) are dissolved in 10 ml DMF, and 0.098 ml of pyridine (96 mg, 1.211 mmol) are added. At 0° C., 0.228 ml of 2-ethylhexyl chloroformate (223 mg, 1.101 mmol, 95% pure) is added dropwise, and the mixture is stirred at 0° C. for 40 min. 20 ml of water are then added, and the reaction mixture is extracted three times with ethyl acetate. The combined organic phases are dried over sodium sulfate and concentrated on a rotary evaporator. The residue is taken up in 25 ml of xylene (isomer mixture) and heated under reflux for 2 days. The mixture is cooled to room temperature and concentrated on a rotary evaporator. The residue is purified by chromatography on silica gel (mobile phase: cyclohexane/ethyl acetate 2:1). This gives 130 mg (38% of theory) of the target compound.

¹H-NMR (400 MHz, DMSO-d₆): δ=1.22-1.39 (m, 4H), 1.43-1.63 (m, 8H), 2.26-2.32 (m, 1H), 4.42 (d, J=7.3, 2H), 7.45 (dd, J=8.1, 4.4, 1H), 8.43 (dd, J=8.1, 1.5, 1H), 8.68 (dd, J=4.4, 1.5, 1H), 13.3 (br. s, 1H).

LC/MS (Method 2): Rₜ=2.41 min.; MS (ESIpos): m/z=314 [M+H]⁺.

### Example 30

### 3-[4-Amino-1-(2-fluorobenzyl)-1H-pyrazolo[3,4-d]pyrimidin-3-yl]-1,2,4-oxadiazol-5(4H)-one

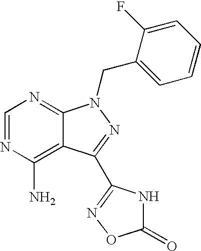

110 mg of 4-amino-1-(2-fluorobenzyl)-N′-hydroxy-1H-pyrazolo[3,4-d]pyrimidine-3-carboximidamide (0.37 mmol, crude product from Example 28A) are dissolved in 2.2 ml of DMF, and 32 μl of pyridine (0.40 mmol) are added. At 0° C., 47 μl of isobutyl chloroformate (0.37 mmol) are added dropwise. The reaction mixture is stirred at 0° C. for 40 min, another 1 eq. of isobutyl chloroformate is then added and the mixture is stirred at 0° C. for another 30 min. The reaction mixture is then stirred at 200° C. in a microwave oven for another 2 h (according to LC/MS, there is still a lot of starting material present). After cooling, ethyl acetate is added to the mixture. The organic phase is washed twice with sodium bicarbonate solution and three times with water and dried over sodium sulfate, and the solvent is removed on a rotary evaporator. As described above, the residue is once more treated with isobutyl chloroformate, and the mixture is then heated in a microwave oven for 2 h. The reaction mixture is then purified directly by preparative HPLC (Method 5). This gives a fraction which is still slightly contaminated and from which some of the desired compound precipitates out. This solid is filtered off and dried under high vacuum. This gives 6 mg (5% of theory) of the title compound.

LC/MS (Method 4): Rₜ=2.01 min.; MS (ESIpos): m/z=328 [M+H]⁺

¹H-NMR (400 MHz, DMSO-d₆): δ=5.68 (s, 2H), 7.11-7.28 (m, 3H), 7.48 (m, 1H), 7.70 (br. s, 1H), 8.35 (br. s, 1H), 8.36 (s, 1H), 13.5 (br. s, 1H).

### Example 31

### 2-[1-(2-Fluorobenzyl)-1H-pyrazolo[3,4-b]pyridin-3-yl]-3,5-dihydro-4H-imidazol-4-one

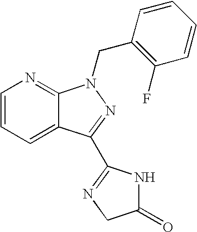

215 mg (0.66 mmol) of the compound from Example 29A in 10 ml of phosphoryl chloride are stirred under reflux for 40 h. The mixture is then concentrated, and the residue is purified by preparative HPLC. This gives 47 mg (22% of theory) of the title compound.

¹H-NMR (400 MHz, DMSO-d₆): δ=4.30 (d, J=5.6 Hz, 2H), 5.84 (s, 2H), 7.12-7.20 (m, 2H), 7.24 (dd, J=10.3, 8.3 Hz, 1H), 7.32-7.40 (m, 1H), 7.46 (dd, J=8.1, 4.4 Hz, 1H), 8.58 (dd, J=8.1, 1.5 Hz, 1H), 8.70 (dd, J=4.4, 1.5 Hz, 1H), 9.17 (t, J=5.6 Hz, 1H).

LC/MS (Method 2): Rₜ=1.92 min.; MS (ESIpos): m/z=310 [M+H]⁺.

### B. ASSESSMENT OF THE PHARMACOLOGICAL ACTIVITY

The pharmacological effect of the compounds according to the invention can be shown in the following assays:

### B-1. Vasorelaxant Effect In Vitro

Rabbits are stunned by a blow to the neck and exsanguinated. The aorta is removed, freed from adhering tissue and divided into rings of a width of 1.5 mm. The rings are placed individually under an initial tension in 5 ml organ baths with Krebs-Henseleit solution which is at 37° C., is gassed with carbogen and has the following composition (in each case mM): NaCl: 119; KCl: 4.8; CaCl₂×2H₂O: 1; MgSO₄×7H₂O: 1.4; KH₂PO₄: 1.2; NaHCO₃: 25; glucose: 10. The force of contraction is detected with Statham UC2 cells, amplified and digitized via A/D converters (DAS-1802 HC, Keithley Instruments, Munich) and recorded in parallel on chart recorders. Contractions are induced by cumulatively adding increasing concentrations of phenylephrine to the bath. After several control cycles, the substance to be investigated is added in each further run in increasing dosage, and the height of the contraction achieved is compared with the height of the contraction reached in the last preceding one. The concentration necessary to reduce the height of the control value by 50% is calculated from this (IC₅₀ value). The standard application volume is 5 μl, the proportion of DMSO in the bath solution corresponds to 0.1%.

Representative IC₅₀ values for the compounds according to the invention are shown in the table below:

| Example No. | IC₅₀ [μM] |
|---|---|
| 1 | 2.1 |
| 2 | 0.45 |
| 3 | 0.61 |
| 6 | 0.95 |
| 7 | 1.1 |
| 18 | 0.61 |
| 19 | 2.1 |

### B-2. Effect on a Recombinant Guanylate Cyclase Reporter Cell Line

The cellular activity of the compounds according to the invention is determined using a recombinant guanylate cyclase reporter cell line, as described in F. Wunder et al., Anal. Biochem. 339, 104-112 (2005).

B-3. Determination of Pharmacokinetic Parameters after Intravenous and Oral Administration

The substance to be examined is administered to animals (for example mice, rats, dogs) intravenously as a solution; oral administration is carried out as a solution or suspension via a stomach tube. After the administration of the substance, blood samples are taken from the animals at predetermined points in time. The blood is heparinized, and plasma is then obtained by centrifugation. The substance is analytically quantified in the plasma by LC/MS-MS. From the plasma concentration/time curves determined in this manner, pharmacokinetic parameters such as AUC, Cₘₐₓ, T_{1/2} (half-life) and CL (clearance) are calculated using a validated pharmacokinetic computer program.

### C. EXEMPLARY EMBODIMENTS OF PHARMACEUTICAL COMPOSITIONS

The compounds according to the invention can be converted into pharmaceutical preparations in the following ways:

### Tablet:

### Composition:

100 mg of the compound according to the invention, 50 mg of lactose (monohydrate), 50 mg of corn starch (native), 10 mg of polyvinylpyrrolidone (PVP 25) (from BASF, Ludwigshafen, Germany) and 2 mg of magnesium stearate.

Tablet weight 212 mg. Diameter 8 mm, radius of curvature 12 mm.

### Production:

A mixture of compound according to the invention, lactose and starch is granulated with a 5% strength solution (m/m) of the PVP in water. The granules are dried and mixed with the magnesium stearate for 5 minutes. This mixture is compressed in a conventional tablet press (see above for format of the tablet). A guideline compressive force for the compression is 15 kN.

Suspension which can be Administered Orally:

### Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound according to the invention.

### Production:

The Rhodigel is suspended in ethanol, and the compound according to the invention is added to the suspension. The water is added while stirring. The mixture is stirred for about 6 h until the swelling of the Rhodigel is complete.

SOLUTION which can be Administered Orally:

### Composition:

500 mg of the compound according to the invention, 2.5 g of polysorbate and 97 g of polyethylene glycol 400.20 g of oral solution correspond to a single dose of 100 mg of the compound according to the invention.

### Production:

The compound according to the invention is suspended in the mixture of polyethylene glycol and polysorbate with stirring. The stirring process is continued until the compound according to the invention has completely dissolved.

i.v. Solution:

The compound according to the invention is dissolved in a concentration below the saturation solubility in a physiologically tolerated solvent (e.g. isotonic saline, 5% glucose solution and/or 30% PEG 400 solution). The solution is sterilized by filtration and used to fill sterile and pyrogen-free injection containers.

1. A compound of the formula (I) 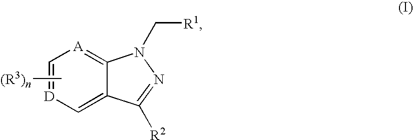
in which
A represents CH, CR³ or N,
D represents CH, CR³ or N if A is N and represents CH or CR³ if A is CH or CR³,
R1 represents phenyl, pyridyl, furyl, thienyl, thiazolyl, oxazolyl, isothiazolyl or isoxazolyl, each of which may be substituted up to two times by identical or different substituents from the group consisting of halogen, cyano, (C₁-C₄)-alkyl, trifluoromethyl and (C₂-C₄)-alkynyl,
or
represents (C₅-C₇)-cycloalkyl which may be substituted up to two times by identical or different substituents from the group consisting of fluorine and (C₁-C₄)-alkyl,
R² represents a group of the formula 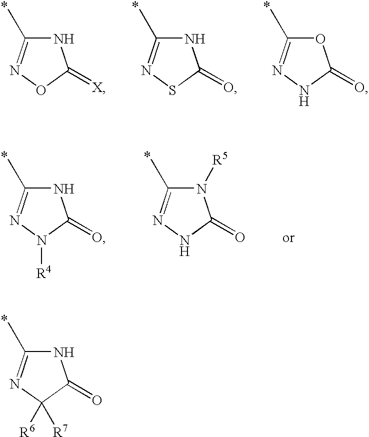
where
* represents the point of attachment to the pyrazole ring,
X represents 0 or S,
R⁴ represents hydrogen, (C₁-C₆)-alkyl or (C₃-C₇)-cycloalkyl,
where (C₁-C₆)-alkyl may be substituted up to five times by fluorine and up to two times by identical or different substituents from the group consisting of (C₃-C₇)-cycloalkyl, hydroxyl, (C₁-C₄)-alkoxy, trifluoromethoxy, (C₁-C₄)-acyloxy, amino, mono-(C₁-C₄)-alkylamino, di-(C₁-C₄)-alkylamino, (C₁-C₄)-acylamino, hydroxycarbonyl, (C₁-C₄)-alkoxycarbonyl, a 5- or 6-membered heterocycle and a group of the formula —C(=0)-NR⁸R⁹, where
R⁸ and R⁹ independently of one another represent hydrogen or (C₁-C₄)-alkyl,
R⁵ represents (C₁-C₄)-alkyl which may be substituted by hydroxyl, (C₁-C₄)-alkoxy, amino, mono-(C₁-C₄)-alkylamino, di-(C₁-C₄)-alkylamino or up to three times by fluorine,
R⁶ has the meaning of R⁴ given above,
R⁷ represents hydrogen or (C₁-C₄)-alkyl
or
R⁶ and R⁷ together with the carbon atom to which they are attached form a spiral-linked 3- to 6-membered cycloalkyl ring,
R³ represents a substituent selected from the group consisting of halogen, cyano, (C₁-C₄)-alkyl, trifluoromethyl, amino, (C₁-C₄)-alkoxy and trifluoromethoxy
and
n represents the number 0, 1 or 2,
where, if the substituent R³ is present more than once, its meanings may be identical or different, or a salt, a solvate or a solvate of a salt thereof.

2. The compound of the formula (I) as claimed in claim 1 in which
A represents N,
D represents CH,
R¹ represents phenyl or thienyl, each of which is mono- or disubstituted by identical or different substituents from the group consisting of fluorine, chlorine, cyano, methyl and trifluoromethyl, or represents cycloheptyl,
R² represents a group of the formula 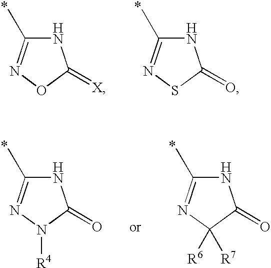
where
* represents the point of attachment to the pyrazole ring,
X represents 0 or S,
R⁴ represents hydrogen or (C₁-C₄)-alkyl,
where (C₁-C₄)-alkyl may be substituted up to five times by fluorine and up to two times by identical or different substituents from the group consisting of (C₃-C₆)-cycloalkyl, hydroxyl, methoxy, ethoxy, trifluoromethoxy, acetoxy, amino, mono-(C₁-C₃)-alkylamino, di-(C₁-C₃)-alkylamino, acetylamino, (C₁-C₄)-alkoxycarbonyl, hydroxycarbonyl, morpholino, piperidino, pyrrolidino and a group of the formula —C(=0)-NR⁸R⁹, where
R⁸ and R⁹ independently of one another represents hydrogen or (C₁-C₄)-alkyl,
R⁶ has the meaning of R⁴ given above
and
R⁷ represents hydrogen,
R³ represents a substituent selected from the group consisting of fluorine, chlorine, methyl, trifluoromethyl, amino, methoxy and trifluoromethoxy
and
n represents the number 0 or 1,
or a salt, a solvate or a solvate of the salt thereof.

3. The compound of the formula (I) as claimed in claim 1 or in which
A represents N,
D represents CH,
R¹ represents phenyl which is mono- or disubstituted by fluorine,
R² represents a group of the formula 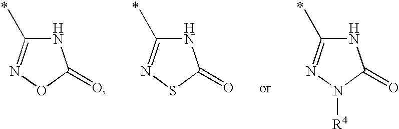
where
* represents the point of attachment to the pyrazole ring
and
R⁴ represents hydrogen or (C₁-C₄)-alkyl which may be substituted up to three times by fluorine
and
n represents the number 0,
or a salt, a solvate or a solvate of the salt thereof.

4. A process for preparing a compound of the formula (I) as defined in claim 1, wherein either
[A] a compound of the formula (II) 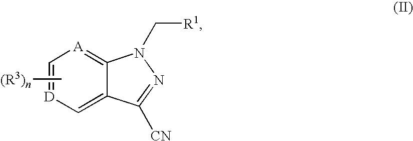
in which A, D, R¹, R³ and n each have the meanings given in claim 1, is initially converted with hydroxylamine into an N′-hydroxyamidine of the formula (III) 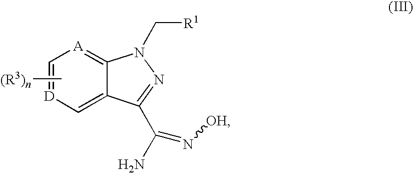
in which A, D, R¹, R³ and n each have the meanings given above,
and this is then converted
[A-1] in the presence of a base with phosgene, a phosgene derivative, such as diphosgene or triphosgene, N,N-carbonyldiimidazole or a chloroformate of the formula (IV) 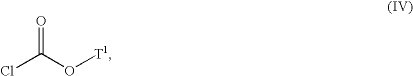
in which
T¹ represents (C₁-C₈)-alkyl,
into a compound of the formula (I-A) 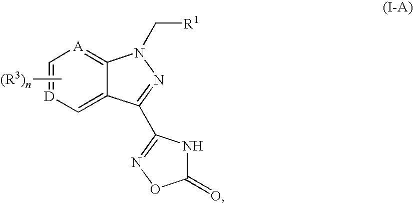
in which A, D, R¹, R³ and n each have the meanings given above,
or
[A-2] in the presence of a base with thiophosgene or N,N′-thiocarbonyldiimidazole into a compound of the formula (I-B) 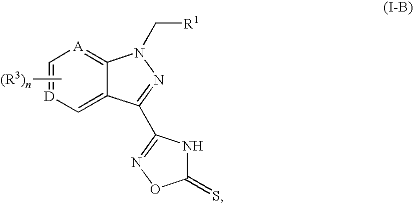
in which A, D, R¹, R³ and n each have the meanings given above,
or
[A-3] initially with N,N′-thiocarbonyldiimidazole and then with boron trifluoride into a compound of the formula (I-C) 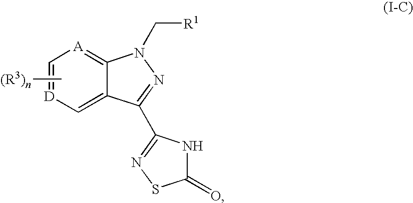
in which A, D, R¹, R³ and n each have the meanings given above
and
[B] a compound of the formula (V) 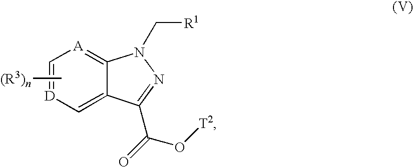
in which A, D, R¹, R³ and n each have the meanings given above
and
T² represents methyl or ethyl,
is initially converted with hydrazine into a compound of the formula (VI) 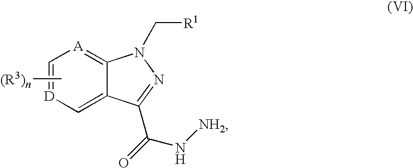
in which A, D, R¹, R³ and n each have the meanings given above,
and this is then converted [B-1] with phosgene, a phosgene derivative, such as diphosgene or triphosgene, or N,N′-carbonyldiimidazole into a compound of the formula (I-D) 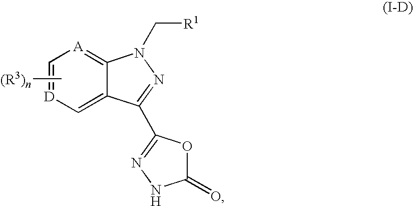
in which A, D, R¹, R³ and n each have the meanings given above,
or
[B-2] with an isocyanate of the formula (VII)
R^{5A}—N═C═O (VII),
in which
R^{5A} has the meaning of R⁵ given in claim 1 or represents 2,4-dimethoxybenzyl,
into a compound of the formula (VIII) 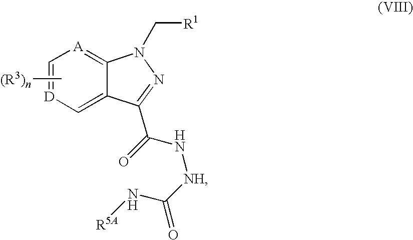
in which A, D, R¹, R³, R^{5A} and n each have the meanings given above,
and then cyclized with the aid of a base to a compound of the formula (I-E) 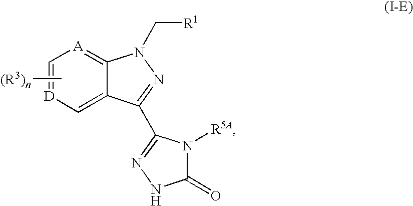
in which A, D, R¹, R³, R^{5A} and n each have the meanings given above, and in the variant where R^{5A} represents 2,4-dimethoxybenzyl, the corresponding compound of the formula (I-E) is then converted in the presence of a base with a compound of the formula (IX)
R^{4A}—X¹ (TX),
in which
R^{4A} has the meaning of R⁴ given in claim 1, but does not represent hydrogen,
and
X¹ represents a leaving group, such as halogen, mesylate, tosylate or triflate,
to a compound of the formula (X) 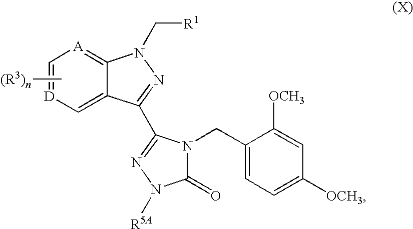
in which A, D, R¹, R³, R^{4A} and n each have the meanings given above,
and the 2,4-dimethoxybenzyl group is then removed with the aid of an acid, giving a compound of the formula (I-F) 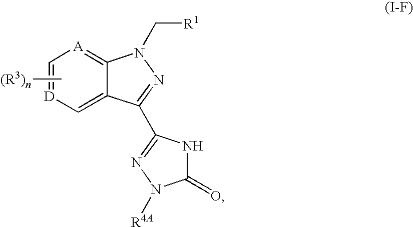
in which A, D, R¹, R³, R^{4A} and n each have the meanings given above,
or
[C] a compound of the formula (XI) 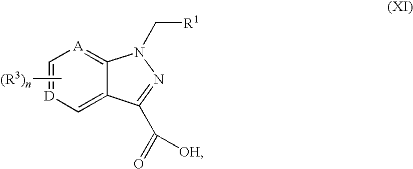
in which A, D, R¹, R³ and n each have the meanings given above,
is initially coupled with a compound of the formula (XII) 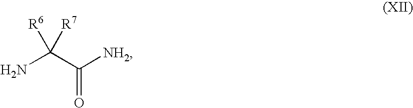
in which R⁶ and R⁷ have the meanings given in claim 1, to give a compound of the formula (XIII) 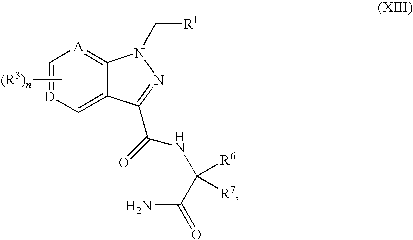
in which A, D, R¹, R³, R⁶, R⁷ and n each have the meanings given above,
and this is then cyclized with phosphoryl chloride to a compound of the formula (I-G) 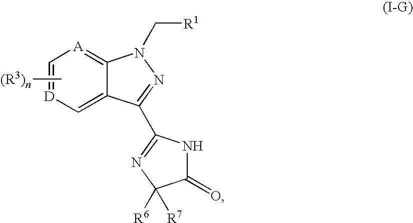
in which A, D, R¹, R³, R⁶, R⁷ and n each have the meanings given above,
and the resulting compounds according to the invention are, if appropriate, converted with the appropriate (i) solvents and/or (ii) acids or bases into their solvates, salts and/or solvates of the salts.

5. A compound of the formula (I) as defined in claim 1 for the treatment and/or prophylaxis of diseases.

6. (canceled)

7. A pharmaceutical composition comprising a compound of the formula (I) as defined in claim 1 in combination with an inert, non-toxic, pharmaceutically suitable excipient.

8. The pharmaceutical composition of claim 7, further comprising an active compound selected from the group consisting of organic nitrates, NO donors, cGMP-PDE inhibitors, agents having antithrombotic activity, agents lowering blood pressure, and agents altering lipid metabolism.

9. The pharmaceutical composition of claim 7 for the treatment and/or prevention of heart failure, angina pectoris, hypertension, pulmonary hypertension, ischemias, vascular disorders, thromboembolic disorders and arteriosclerosis.

10. A method for the treatment and/or prevention of heart failure, angina pectoris, hypertension, pulmonary hypertension, ischemias, vascular disorders, thromboembolic disorders and arteriosclerosis in humans and animals by administration of an effective amount of at least one compound of the formula (I) as defined in claim 1.

11. A method for the treatment and/or prevention of heart failure, angina pectoris, hypertension, pulmonary hypertension, ischemias, vascular disorders, thromboembolic disorders and arteriosclerosis in humans and animals by administration of an effective amount of a pharmaceutical composition of claim 7.
